(19) United States
(12) Patent Application Publication
(10) Pub. No.: US 2026/0153517 A1
(43) Pub. Date: Jun. 4, 2026

(54) REPORTER CELLS EXPRESSING CHIMERIC POLYPEPTIDES FOR USE IN DETERMINING PRESENCE AND OR ACTIVITY OF RECEPTORS ASSOCIATED WITH CANCER AND SELECTING TREATMENT

(71) Applicant: B.G. Negev Technologies and Applications Ltd., Beer-Sheva (IL)

(72) Inventors: Angel PORGADOR, LeHavim (IL); Moshe ELKABETS, Beit Kama (IL)

(73) Assignee: B.G. Negev Technologies and Applications Ltd., Beer-Sheva (IL)

(21) Appl. No.: 19/111,805

(22) PCT Filed: Sep. 14, 2023
(86) PCT No.: PCT/IL2023/051010

§ 371 Date: Mar. 14, 2025

Related U.S. Application Data
(60) Provisional application No. 63/406,303, filed on Sep. 14, 2022.

(51) Int. Cl. G01N 33/68; C07K 16/30; G01N 33/50; G01N 33/575

(52) U.S. Cl. CPC G01N 33/6893; C07K 16/30; G01N 33/5091; G01N 33/5759; G01N 2333/71; G01N 2333/715; G01N 2800/52; G01N 2800/60; G01N 2800/7028

(57) ABSTRACT

A polynucleotide encoding a chimeric polypeptide comprising an amino acid sequence of a ligand capable of binding a cancer-associated receptor is provided. The amino acid sequence of the ligand being translationally fused to a heterologous amino acid sequence of a cell signaling module such that upon binding of the amino acid sequence of the ligand to the receptor, the cell signaling module is activated, wherein the ligand or cancer-associated receptor is not an immune checkpoint molecule.

### RELATED APPLICATIONS

This application claims priority from U.S. Provisional Patent Application No. 63/406,303 filed Sep. 14, 2022, which is hereby incorporated by reference in its entirety.

### SEQUENCE LISTING STATEMENT

The XML file, entitled 96740 Sequence Listing.xml, created on Sep. 14, 2023, comprising 70,264 bytes, submitted concurrently with the filing of this application is incorporated herein by reference.

### FIELD AND BACKGROUND OF THE INVENTION

The present invention, in some embodiments thereof, relates to reporter cells expressing chimeric polypeptides for use in determining presence and or activity of receptors associated with cancer and selecting treatment.

Personalized cancer therapy is based on the precept that detailed molecular characterization of the patient's tumor and its microenvironment will enable tailored therapies to improve outcomes and decrease toxicity. The goal of personalized therapy is to target aberrations that drive tumor growth and survival, by administering the right drug combination for the right person. This is becoming increasingly achievable with advances in high-throughput technologies to characterize tumors and the expanding repertoire of molecularly targeted therapies. However, there are numerous challenges that need to be surpassed. These include tumor heterogeneity and molecular evolution, costs and potential morbidity of biopsies, lack of effective drugs against most genomic aberrations, technical limitations of molecular tests, and reimbursement and regulatory hurdles.

### RELATED BACKGROUND ART

Ramirez-Chacon A, Betriu-Mendez S, Bartolo-Ibars A, Gonzalez A, Marti M and Juan M (2022) Ligand-based CART cell: Different strategies to drive T cells in future new treatments. Front. Immunol. 13:932559.

### SUMMARY OF THE INVENTION

According to an aspect of some embodiments of the present invention there is provided a method of detecting presence and/or activity of a cancer-associated receptor in a cancer cell, the method comprising:
(a) contacting the cancer cell with a reporter cell comprising a polynucleotide encoding a chimeric polypeptide comprising an amino acid sequence of a ligand capable of binding a cancer-associated receptor, the amino acid sequence of the ligand being translationally fused to a heterologous amino acid sequence of a cell signaling module such that upon binding of the amino acid sequence of the ligand to the receptor, the cell signaling module is activated, wherein the ligand or cancer-associated receptor is not an immune checkpoint molecule;
(b) determining activation of the cell signaling module in the reporter cell, the activation being indicative of the presence and/or activity of the cancer-associated receptor or dimers or heterodimers thereof in the cancer cell.

According to an aspect of some embodiments of the present invention there is provided a polynucleotide encoding a chimeric polypeptide comprising an amino acid sequence of a ligand capable of binding a cancer-associated receptor, the amino acid sequence of the ligand being translationally fused to a heterologous amino acid sequence of a cell signaling module such that upon binding of the amino acid sequence of the ligand to the receptor, the cell signaling module is activated, wherein the ligand or cancer-associated receptor is not an immune checkpoint molecule.

According to an aspect of some embodiments of the present invention there is provided a nucleic acid expression construct comprising a nucleic acid sequence encoding the polynucleotide of claim 1 under transcriptional control of a cis-acting regulatory element(s).

According to an aspect of some embodiments of the present invention there is provided a reporter cell comprising the polynucleotide of claim 1 or the nucleic acid construct as described herein.

According to an aspect of some embodiments of the present invention there is provided a method of detecting presence and/or activity of a cancer-associated receptor in a cancer cell, the method comprising:
(a) contacting the cancer cell with the reporter cell as described herein;
(b) determining activation of the cell signaling module in the reporter cell, the activation being indicative of the presence and/or activity of the cancer-associated receptor or dimers or heterodimers thereof in the cancer cell.

According to an aspect of some embodiments of the present invention there is provided a method of treating a subject diagnosed with cancer, the method comprising:
(a) detecting presence and/or activity of a cancer-associated receptor in a cancer cell of the subject as described herein; and
(b) treating the subject with an inhibitor of the receptor when presence or a predetermined threshold of activity of the cancer-associated receptor or dimers or heterodimers thereof is indicated or with another treatment modality when it is not indicated or absent.

According to an aspect of some embodiments of the present invention there is provided a method of selecting treatment for a subject diagnosed with cancer, the method comprising:
(a) detecting presence and/or activity of a cancer-associated receptor in a cancer cell of the subject as described herein; and
(b) selecting treatment for the subject with an inhibitor of the receptor when presence or a predetermined threshold of activity of the cancer-associated receptor or dimers or heterodimers thereof is indicated or with another treatment modality when it is not indicated or absent.

According to some embodiments of the invention, the inhibitor is a chimeric antigen receptor (CAR).

According to some embodiments of the invention, the CAR is an effector immune cell expressing the polynucleotide encoding a chimeric polypeptide as described herein.

According to some embodiments of the invention, the chimeric polypeptide is encoded by the polynucleotide as described herein.

According to some embodiments of the invention, the cancer associated receptor is selected from the group consisting of a growth factor receptor, a cytokine receptor and a chemokine receptor.

According to some embodiments of the invention, the cancer-associated receptor is selected from the group of ErbB-1, ErbB-2, ErbB-3 and ErbB-4.

According to some embodiments of the invention, the ligand is selected from the group consisting of NRG-1 alpha (NRG-1A), NRG-1 (NRG-1B) beta and betacellulin (BTC).

According to some embodiments of the invention, the detecting presence and/or activity of a cancer-associated receptor in a cancer cell comprises detecting presence and/or activity of plurality of cancer-associated receptors using a plurality of reporter cells of claim 3 each expressing at least one distinct chimeric polypeptide which binds the receptor or dimers or heterodimer thereof.

According to some embodiments of the invention, the cancer-associated receptor is vascular endothelial growth factor receptor.

According to some embodiments of the invention, the ligand is VEGFA.

According to some embodiments of the invention, the cell signaling module comprises a transmembrane domain and/or a cytoplasmic portion of a cell signaling receptor.

According to some embodiments of the invention, the cell signaling module comprises a cytoplasmic portion of a cell signaling receptor.

According to some embodiments of the invention, the cell signaling module comprises a transmembrane domain and/or a cytoplasmic portion of a receptor kinase.

According to some embodiments of the invention, the receptor kinase is a tyrosine kinase or serine/threonine kinase.

According to some embodiments of the invention, the cell signaling module comprises an adaptor molecule.

According to some embodiments of the invention, the cell signaling module comprises a CD3 zeta chain.

According to some embodiments of the invention, activation of the cell signaling module is by dimerization, oligomerization and/or post-translational modification.

According to some embodiments of the invention, the determining activation is by analyzing a cytokine and/or an interleukin induced by the activation.

According to some embodiments of the invention, the interleukin is selected from the group consisting of IL-2 and IL-8.

According to some embodiments of the invention, the determining activation is by analyzing a phenotype selected from the group consisting of proliferation, apoptosis, migration, post-translational modification, biomolecule expression, biomolecule secretion, morphology and cell cycle distribution.

According to some embodiments of the invention, the cell is an immune cell.

According to some embodiments of the invention, the cell is a non-cancerous cell.

According to some embodiments of the invention, the cell is a transgenic cell.

According to some embodiments of the invention, the cell is null for expression of the cancer-associated receptor and/or ligand.

According to some embodiments of the invention, the cell is transformed to express a fluorescent or bioluminescent molecule upon activation of the cell signaling module.

According to some embodiments of the invention, the contacting is in the presence of an inhibitor of the cancer-associated receptor.

According to some embodiments of the invention, the inhibitor is selected from the group consisting of an antibody, an aptamer and a peptide.

According to some embodiments of the invention, the cancer cell is comprised in a tissue biopsy.

According to some embodiments of the invention, the tissue biopsy is fresh.

According to some embodiments of the invention, the tissue biopsy is fixated.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are a schematic illustration of a model for assessing receptor availability and homo/hetero dimers status on cancer cells. (A) Description of 4 BW-expressed reporters including the active part of EGF-like ligands fused to zeta. Upon fitted binding of the ligand to HER based homo/hetero dimers on target cancer cells, IL-2 is secreted from the BW reporter and the intensity of IL-2 secreted represents the efficacy of ligand binding to the various homo/hetero dimers on cancer cells. (B) A literature-based predicted pattern of ligand recognition of various homo/hetero HER dimers, and in accordance, intensity of IL-2 secretion by the 4 reporters (sH1, HER1 homodimer; sH2, HER2 homodimer, sH3, HER3 homodimer; sH4, HER4 homodimer; dH12, HER1-HER2 hetero dimer; dH23, HER2-HER3 heterodimer; dH24, HER2-HER4 heterodimer). Note that IL-2 secretion is based on the ligand-fused-zeta mediated signal in the reporter and is representing the presence and dimer status of the HER family receptors on target cells.

FIGS. 2A-F are FACS analysis images in Wild Type JIM T1 cells and in JIM T1 cells that were knocked out for HER2 expression. (A-C) Expression of HER1, HER2, HER3 in JIM T1 WT cells respectively. (D-F) Expression of HER1, HER2, HER3 in JIM T1 KO cells respectively. JIM T1 expresses all the HERs, while a significant reduction in HER2 expression in the JIM T1 KO can be observed.

FIG. 3 shows IL-2 based response of the artificial reporters HERDET1 and HERDET 2 to JIMT1 and HER2-KO JIMT1 cells. Reduction of response is strong for HERDET1 when compared between JIMT1 and JIMT1-KO, while the reduction in response of HERDET2 is very moderate when comparing JIMT1 and JIMT1-KO targets. This suggests that HERDET1 is more specific to the heterodimer HER2+HER3 while HERDET2 recognizes both the homodimer HER3 and the heterodimer HER2+HER3.

FIG. 4 is an illustrative embodiment for clinically assaying HER receptors on cancer cells.

FIG. 5 shows sequences of HER-ligand reporter molecules according to some embodiments of the invention;

FIGS. 6A-B show an IL-2 based response of BW-VEGFA to A549 cells (A-50,000 A549 cells; B-25,000 A549 cells). In both the combinations, Avastin® reduced the response of IL-2 in BW-VEGFA cells.

FIG. 7 shows a sequence of a VEGFA reporter molecule according to some embodiments of the invention;

FIGS. 8A-D are illustrative figures for reporter molecules according to some embodiments of the invention. FIG. 8A-shows the core ligand domain of NRG1a, NRG1b, and BTC (SEQ ID NO: 50, 51 and 52 of HERDET1, HERDET2 and HERDET10, respectively). FIG. 8B shows the construction of reporter polypeptides according to some embodiments of the invention. The sequences comprise selected amino acids of the ligands followed by (G4S) 4 as a linker, MYC, human CD8, murine CD3 zeta transmembrane domain, and murine CD3 zeta cytoplasmic domain (HERDET1, HERDET2 and HERDET10). FIG. 8C shows the detection of HER family homodimers/heterodimers based on mIL-2 secretion upon binding to target cancer cells/BW standards. FIG. 8D shows the generation of BW standards expressing HER1, HER2, HER3, HER1+2, HER1+3, and HER2+3.

FIGS. 9A-G show the selection of positive reporters by flow cytometry using α-MYC staining. FIG. 9A—NRG1a without puromycin selection; FIG. 9B—NRG1a after puromycin selection; FIG. 9C—NRG1b without puromycin selection; FIG. 9D—NRG1b after puromycin selection; FIG. 9E—BTC without puromycin selection; FIG. 9F—BTC after puromycin selection; FIG. 9G—ELISA for mIL-2 secretion from BW-reporters after binding to α-MYC antibody coated on plastic.

FIGS. 10A-F show the selection of BW standards (i.e., receptors) by flow cytometry using α-MYC staining. FIG. 10A—W-HER1 with antibiotic selection; FIG. 10B—BW-HER2 with antibiotic selection; FIG. 10C—BW-HER3 with antibiotic selection; FIG. 10D—BW-HER1+3 with antibiotic selection; FIG. 10E—BW-HER1+2 with antibiotic selection selection; FIG. 10F—BW-HER2+3 with antibiotic selection

FIG. 11 shows secretion of mIL-2 in BW standards following reporter binding.

FIGS. 12A-C shows HER expression profile in BW standards. FIG. 12A—HER1 expression profile in BW-HER1, BW-HER1+2, BW-HER1+3; FIG. 12B—HER2 expression profile in BW-HER2, BW-HER1+2, BW-HER2+3; FIG. 12C—HER3 expression profile in BW-HER3, BW-HER1+3, BW-HER2+3

FIGS. 13A-D show the response of BW reporters to cancer cells. FIG. 13A—Flow cytometry analysis for HER1,2,3, expression in Cal33 (WT and KD), JIMT1 (WT and KO), and A375; FIG. 13B—ELISA for NRG1a, NRG1b, BTC and NORM against Cal33 (WT and KD), JIMT1 (WT and KO), and A375; FIG. 13C—A table for HER expression profile in cancer cell lines; FIG. 13D—A table for BW reporter binding to HERs.

### DESCRIPTION OF SPECIFIC EMBODIMENTS OF THE INVENTION

The present invention, in some embodiments thereof, relates to reporter cells expressing chimeric polypeptides for use in determining presence and or activity of receptors-associated with cancer and selecting treatment.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Personalized medicine is rooted in the belief that since individuals possess nuanced and unique characteristics at the molecular, physiological, environmental exposure and behavioral levels, they may need to have interventions provided to them for diseases they possess that are tailored to these nuanced and unique characteristics. This belief has been verified to some degree through the application of emerging technologies such as DNA sequencing, proteomics, imaging protocols, and wireless health monitoring devices, which have revealed great inter-individual variation in disease processes.

Whilst conceiving embodiments of the invention and reducing them to practice, the present inventors configured a cell-based reporter system that can recognize the expression and availability of cancer-associated receptors on cancer cells and as such can be used in predicting disease outcome and selecting specific treatment modalities. In one embodiment, this system is referred to as “Targeted chemotherapy Artificial Reporter (TcAR)”.

Some utilities of the system are provided in the Examples section which follows. Example 1-shows the efficacy of the system in assessing highly complexed receptor presentation/activity on cancer cells. Specifically, a receptor family associated with cancerous state/cancer progression for which (i) homo/hetero dimerization forms exist; (ii) multiple ligands exist and with various affinities to different receptor forms; and (iii) clinically-employed drugs exist with distinct efficacies to the various homo/hetero forms. Using the system in which a plurality of reporter cells function as an E-nose it is possible to assess the homo/hetero dimer status of the receptors on the cancer cell, by employing various artificial receptor-ligands and calculating the relative intensity of the reporter response. This is exemplified on the HER (ErbB) family which includes 4 receptors, 11 ligands and a plurality of homodimers and heterodimers, each being induced by different interactions. This approach can be used to diagnose, prognose cancer in patients, select treatment modalities (only for the type of ligand-blocking drugs, e.g., antibodies) in an individualized manner and search for new drugs.

In another example, the system is used to evaluate the functional bioavailability of receptors on the cancer cell, by employing Ligand-expressing reporters and clinical drug-based scoring (only for the type of ligand-blocking drugs, e.g., antibodies). This is shown in Example 2 as implemented in a VEGFA-expressing AR and the Avastin® (mAb anti-VEGFA) drug.

It is expected that the newly devised reporter system will have significant contribution to cancer patients, such that determining receptor expression and availability for treatment becomes standard for predicting disease outcome and selection of treatments.

Thus, according to an aspect of the invention there is provided a polynucleotide encoding a chimeric polypeptide comprising an amino acid sequence of a ligand capable of binding a cancer-associated receptor, said amino acid sequence of said ligand being translationally fused to a heterologous amino acid sequence of a cell signaling module such that upon binding of the amino acid sequence of said ligand to said receptor, said cell signaling module is activated, wherein said ligand or cancer-associated receptor is not an immune checkpoint molecule.

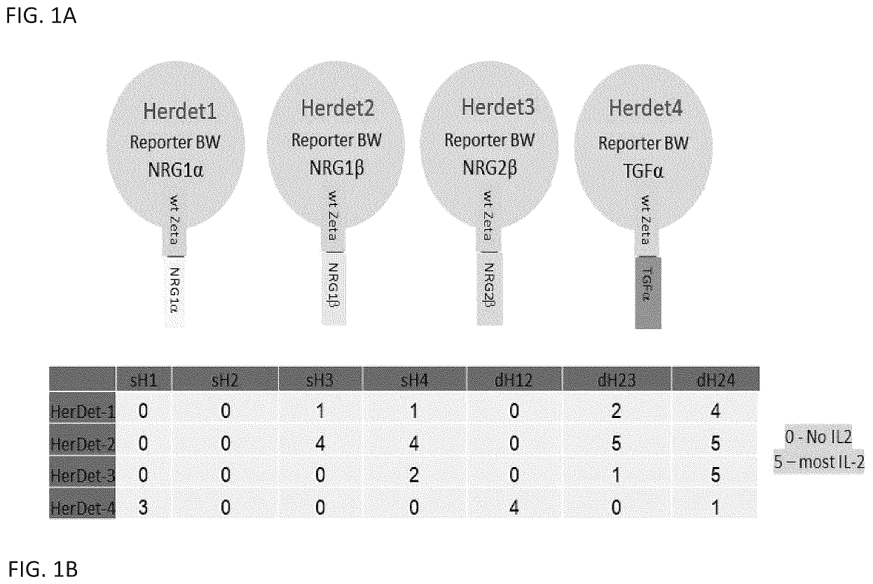

It will be appreciated that according to some embodiments, especially when there is more than one ligand to a specific receptor or when ligand binding necessitates heterodimerization, the use of several chimeric polypeptides and reporter cells expressing same (also referred to herein as a “clinical array”) is contemplated (FIGS. 1A-B and 4).

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above). According to a specific embodiment, the polynucleotide is dsDNA.

As used herein “a chimeric polypeptide” or “fusion polypeptide” refers to a polypeptide in which proteinaceous components which are not found in nature on a single polypeptide or at the same orientation on a single polypeptide are fused, typically covalently and preferably by a peptide bond. Thus, the proteinaceous components are heterologous to one another. The chimeric polypeptide is presented on a cell membrane in the following general orientation from N to C: extracellular ligand portion-transmembrane domain and a cytoplasmic portion. Intervening sequences can be included anywhere in the chimeric polypeptide.

As used herein, the term “heterologous” refers to an amino acid sequence which is not native to the recited amino acid sequence at least in localization or is completely absent from the native sequence of the recited amino acid sequence.

The components can be linked directly or via a linker (e.g., amino acid linker, e.g. GGGGSGGGGSGGGGSGGGGS), and/or a “hinge” region (e.g., amino acid hinge, e.g. CD8a hinge region) that is located between the transmembrane domain and recognition moiety.

Non-limiting examples of polypeptide linkers include linkers having the sequence LE, GGGGS (SEQ ID NO: 1), (GGGGS)ₙ (n=1-4) (SEQ ID NO: 2), GGGGSGGGG (SEQ ID NO: 3), (GGGGS)x2 (SEQ ID NO: 4), (GGGGS)x2+GGGG (SEQ ID NO: 5), (Gly) 8 (SEQ ID NO: 15), (Gly) 6 (SEQ ID NO: 16), (EAAAK), (n=1-3) (SEQ ID NO: 6), A(EAAAK)ₙA (n=2-5) (SEQ ID NO: 7), AEAAAKEAAAKA (SEQ ID NO: 8), A(EAAAK)₄ALEA(EAAAK)4A (SEQ ID NO: 9), PAPAP (SEQ ID NO: 10), KESGSVSSEQ LAQFRSLD (SEQ ID NO: 11), EGKSSGSGSESKST (SEQ ID NO: 12), GSAGSAAGSGEF (SEQ ID NO: 13), GGGGSGGGGSGGGGSGGGGS (SEQ ID NO: 14).

According to some embodiments the linker is as set forth in GGGGSGGGGSGGGGSGGGGS (SEQ ID NO: 14).

As used herein “cancer-associated receptor” refers to a cell surface receptor, which expression is associated with cancer onset or progression. Such a receptor typically comprises or is associated with a cytoplasmic cell signaling module which typically mediates cell proliferation, cell differentiation, resistance to drugs, cell mobilization and many more biological processes.

According to a specific embodiment, the cancer-associated receptor is a growth factor receptor or a family or growth factor receptors.

Growth factor receptors (GFRs), expressed on cell membranes, have profound roles in cell growth, survival, angiogenesis and metastasis. Amplification of GFRs generates inherent and acquired resistance to classical chemotherapies and targeted molecules. Escalated growth signals cross-talk differently with death signals to inhibit apoptosis that is programmed cell death. Accordingly, signals mediated by GFRs function in collaboration to enhance the complexity of the tumor microenvironment.

According to a specific embodiment, the cancer associated receptor is selected from the group consisting of a growth factor receptor, a cytokine receptor and a chemokine receptor.

According to a specific embodiment, the cancer-associated receptor is a receptor tyrosine kinase (RTK, E.C. 2.7.10.1) or a serine/threonine kinase (E.C. 2.7.11).

The reporter cells express the chimeric polypeptide in which the ligand portion is selected according to the identity of the receptor which expression/activity/availability is assayed. The skilled in the art of signaling would know which pairs or higher ordered combinations to select.

Following is a non-limiting review of some cancer-associated receptor families and their ligands.

According to a specific embodiment, the receptor is of the epidermal growth factor receptor family.

The epidermal growth factor receptor (EGFR) family encompasses four receptor proteins, namely ErbB-1/EGFR-1 to -4 (also called HER 1-4) that are expressed on cell surface and exhibit tyrosine kinase activities. These proteins have similar structures and are comprised of three domains: an extracellular domain with ligand binding site, a transmembrane domain, and an intracellular domain with kinase activity. There are 11 different growth factors, each possessing a conserved EGF domain that can bind with those four receptors. Upon ligand binding, the receptors form homo- or hetero-dimers, promoting activation, relaying signals for proliferation, survival, migration and differentiation and thus playing major roles in cancer progression. Overexpression and/or gene amplification of EGFR confer malignancy to diverse tissues. Moreover, constitutively active mutants of EGFR are found in different cancers, where they are often associated with poor prognosis.

The ability of the proteins in this family to form dimers and heterodimers affect their functionality. Hence it is important to assess not only each receptor individually but their ability to form homodimers and heterodimers. For instance, the heterodimer of ErbB-2/ErbB-3 is particularly oncogenic and hence determining its presence and/or level is of high significance.

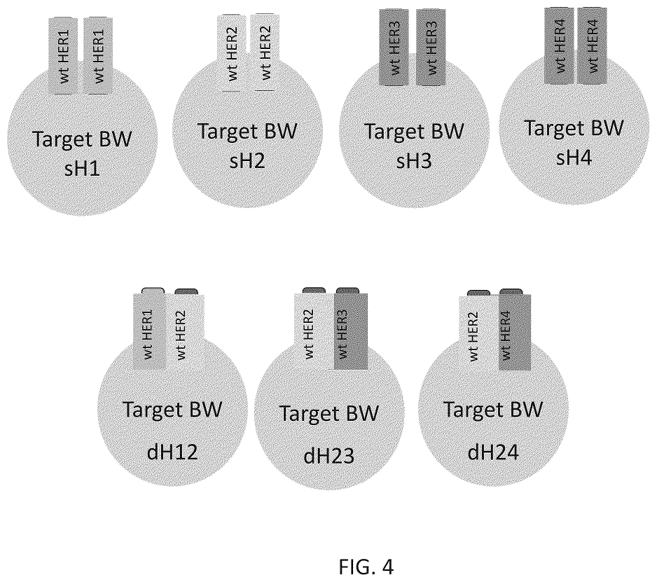

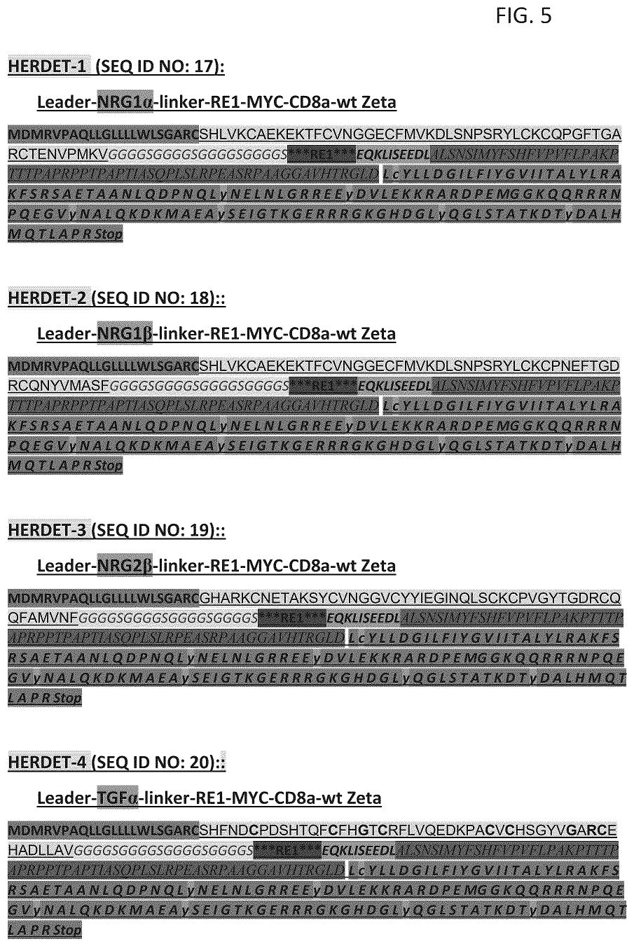

FIGS. 1A-B describe the principle of determining ErbB profile on cancer cells using the chimeric polypeptides expressed in reporter cells and referred to herein as “Herdet”. FIG. 4 describes an exemplary set of a reporter cell panel for the clinical assessment of clinical samples (e.g., tissue biopsies).

According to a specific embodiment, the receptor is of the insulin-like growth factor receptor family.

The insulin-like growth factor receptor (IGFR) family consists of two cell membrane receptors, IGF1R and IGF2R. IGF1R (that also forms a heterodimer with the insulin receptor [IR]) binds to insulin-like growth factor 1 (IGF1) with higher affinity and IGF2 with comparatively lower affinity to elicit the growth signals required for foetal and postnatal development. The post-translationally modified IGF1R is a polypeptide containing one α- and one β-chain that are connected by a disulfide bond and expressed on the cell surface. The α-chain and portion of the β-chain comprise the extracellular domain followed by transmembrane and cytoplasmic domain in β-chain. The mature IGF1R is a homodimer comprising the α2 and β2 chains linked by disulfide bonds. The intracellular domain has tyrosine kinase activity that auto-phosphorylates the receptor and a number of downstream proteins upon binding to the ligands. The notion of involvement of this receptor in tumorigenesis came from the studies of IGF1R-transfected cells and the effects of IGF1R gene mutation. Overexpression of IGF1R gene is implicated in cellular proliferation, transformation, and metastasis in several carcinomas. Amplification of IGF1R gene in breast cancer and melanoma and overexpression of IGF1R gene in pediatric cancer has been reported.

The relatively simpler IGF2R (also called mannose-6 phosphate receptor, M6P) comprises a single polypeptide chain, and functions as a “scavenger receptor” for IGF2. It suppresses tumor growth, modulates invasiveness, and blocks angiogenesis. Mutations in IGF2R locus have been observed in lung cells and identified as an early event in hepatocellular carcinoma in different populations.

According to a specific embodiment, the receptor is of the transforming growth factor-beta receptor family.

The transforming growth factor-beta receptor (TGF-βR) family comprises three membrane receptors (TβRI, TβRII and TβRIII) which are expressed in diverse types of cells and regulate distinct cellular functions by the signals transduced upon TGF-β ligand binding. TβR and TβRII are single pass serine/threonine kinases with N-terminal ectodomains and C-terminal kinase domains. TβRIII (also known as betaglycan) is a cell surface proteoglycan >300 kDa in molecular mass and does not possess an intracellular kinase domain. TβIII binds with TGF-β ligands and presents them to TβRII or the ligands bind directly with TβRII depending on cell types. After binding, TβRII recruits and trans-phosphorylates TβRI, which in turn activates SMAD proteins. SMAD complexes translocate into the nucleus and function as transcription factors for TGF-β responsive genes and thus regulate cell proliferation, survival, migration and differentiation. TGF-βR-mediated signals play context-dependent dual roles in cell growth. Under physiological conditions, TGF-β prevents cell growth, stimulates apoptosis or differentiation. During tumorigenesis, TGF-βR-mediated signals promote cell growth due to genetic and epigenetic changes. Mutations and dis-regulation of TGF-βR genes were observed in different cancers, for example, down-regulation of TGF-βRII gene in breast and lung cancer and different mutations in colon and pancreatic cancer.

According to a specific embodiment, the receptor is of the vascular endothelial growth factor receptor family.

This family consists of three membrane receptors (VEGFR1-3), predominantly expressed on endothelial cells and few additional cell types. VEGFRs are single pass protein with seven immunoglobulin (Ig)-like domains on the extracellular site and two split tyrosine kinase domains in the intracellular site. They bind with the disulfide-linked homodimer of VEGF isoform (VEGFA-D) ligands and placenta growth factors (PIGF1 and 2) to form homodimers or heterodimers of VEGFR-1 and -2 and relay the signal inside cells. The signals transduced by VEGFR are different between these receptors. For example, VEGFR2 (also known as KDR/flk-1) induces mitogen-activated protein kinases (MAPK)-dependent cell proliferation whereas VEGFR1 (flt-1) does not induce cell growth. However, activation of VEGFR1 by VEGF stimulates cell migration, a response that is also triggered by VEGFR2 activation. These VEGF-VEGFR interactions are well-known for their key roles in vasculogenesis and angiogenesis. VEGFR3 (flt-4) that is expressed on lymphatic vessels interacts with VEGF-C and VEGF-D and is thought to promote lymphangiogensis. VEGFRs are thought to be responsible for blood and lymph vessel formation in tumor microenvironment and thus promote tumor growth and progression. High expression of VEGFR gene is observed in many different types of malignancies. Moreover, somatic mutations in VEGFR2 and VEGFR3 genes were identified in the most common infants' malignancy, juvenile hemangioma.

Numerous treatment modalities directed at this family may gain from using the present platform as exemplified in Example 2 below.

According to a specific embodiment, the receptor is of the platelet derived growth factor receptor family.

The platelet-derived growth factor receptor (PDGFR) family contains two receptors (PDGFR-α and -β) that are encoded by two different genes and are expressed on the membrane of different cell types. These single chain receptor proteins have five Ig-like extracellular domains and a tyrosine kinase domain. Dimerization of receptors occurs upon binding to homo/heterodimers of PDGF (A-D) ligands, leading to conformational changes in receptors, activating them to trans-phosphorylate and stimulate downstream proteins. This relays the signals into receiving cells via mainly MAPK and PI3K pathways and thus regulates cell proliferation, differentiation, growth, migration, and survival. They have roles in angiogenesis and thus support tumor growth. Overexpression and mutations in the PDGFR genes are associated with diverse cancers. Aberrant expression of PDGFR due to amplification and/or overexpression of PDGFRα and PDGFRβ genes were reported in human glioblastoma multiforme. Moreover, mutations and genetic translocation in PDGFRα gene were observed in gastrointestinal stromal tumors and chronic leukemia respectively. A germline point mutation (gain of function) in PDGFRβ gene was found in the most common fibrous tumor of infancy, myofibromatosis.

According to a specific embodiment, the receptor is of the fibroblast growth factor receptor family.

The fibroblast growth factor receptor (FGFR) family consists of four closely related transmembrane proteins (FGFR1-4) and their different isoforms with altered ligand specificity due to differential splicing of FGFR mRNA. These single chain receptors contain one extracellular domain with three immunoglobulin repeats (Ig I-III) with ligand binding capacity, one transmembrane domain and one intracellular domain with kinase activity at the carboxy-terminus. There are 18 different FGF ligands that can bind to different FGF receptors. Upon binding, dimerization of FGFR leads to auto-phosphorylation and kinase activation. Phosphorylated FGFRs in turn phosphorylate a number of proteins and/or serve as molecular docking sites for many effectors, thus orchestrating context-dependent cellular functions including cell proliferation, growth, differentiation, migration, vascular repair, wound healing, and cell survival. FGF-FGFR interactions have pivotal roles in tumorigenesis as the downstream mitogenic growth signals (MAPK) and anti-apoptotic PI3K/AKT signals lead to uncontrolled growth and inhibition of cell death, respectively. The PLC/PKC pathway downstream of FGFRs also converges to the MAPK pathway to support cell growth. These receptors have been shown to exert profound roles in angiogenesis both in paracrine and autocrine fashions. FGFR expression causes tumor cells to acquire resistance to several drugs, especially inhibitors targeting other growth factor receptors (EGFR, PDGFR and VEGFR) because of their extensive cross-talks. Amplification and mutations in FGFR genes that lead to constitutive activation/up-regulation of receptors are found in different types of malignancies, including breast, ovarian, gastric and lung cancers.

More examples are provided in Tables 1-3 below.

TABLE 1

| Receptor | Ligands |
|---|---|
| ErbB-1, ErbB-2, | EREG, EGF, TGFa, HB-EGF, NRG1A, NRG1B, |
| ErbB-3, ErbB-4 | NRG2A, NRG2B, AREG, BTC, NRG3, NRG4 |
| IGF1R | IGF1 IGF2 |
| InsR | Insulin |
| PDGFR a/b | PDGF A/B/C/D |
| KIT/SCFR | SCF FLt3L |
| FGFR1/2/3/4 | FGF 1-23 |
| Trk A/B/C | NGF, BDNF, NT3, NT4 |
| MET | HGF, MSP |
| AXL/mer/Tyro3 | GAS6, Protein S |
| Tie 1/2 | Ang1-4 |
| DDR1/2 | Collagens |
| RET | GDNF, GDF15, Artemin |
| ALK | ALKL1/2 |
| VEGFR1/2/3 | VEGFA/B/C/D |

TABLE 2

| Receptor | Ligands |
|---|---|
| CXCR1 | IL-8. GCP2 |
| CXCR4 | SDF1 |
| CXCR5 | BCA1 |
| CCR1 | MIP1a Rantes MCP3 |
| CCR2 | MCP1, 2, 4 |
| CCR5 | MIP1a Rantes MIP1b |
| XCR1 | XCL1 |

TABLE 3

| Receptor | Ligands |
|---|---|
| IFNGR1 | IFNg |
| INFGR2 | IFNb IFNa |
| IL10R | IL-10 |
| IL17R | IL17 |

According to a specific embodiment, the cancer-associated receptor is selected from the group of ErbB-1, ErbB-2, ErbB-3 and ErbB-4.

According to a specific embodiment, the ligand is selected from the group consisting of EGF, amphiregulin, betacellulin, epigen, epiregulin, HB-EGF, TGFα, NRG-1 (e.g., NRG-1A AND NRG-1B), NRG-2, NRG-3 and NRG-4 (see e.g., Example 1 with the Herdet examples).

According to a specific embodiment, the ligand is selected from the group consisting of NRG-1 alpha (NRG-1A), NRG-1 (NRG-1B) beta and betacellulin (BTC).

As used herein “NRG1” is characterized as HGCN 7997, NCBI gene 3084, Ensembl, OMIM and UniprotKB.

As used herein “NRG2” is characterized as HGCN 7998, NCBI gene 9542, Ensembl, OMIM and UniprotKB.

As used herein “NRG3” is characterized as HGCN 7999, NCBI gene 10718, Ensembl, OMIM and UniprotKB.

As used herein “NRG4” is characterized as HGCN 29862, NCBI gene 145957, Ensembl, OMIM, UniprotKB.

As used herein “BTC” is characterized as HGCN 1121, NCBI gene 685, Ensembl ENSG000001748208, OMIM, UniprotKB.

As used herein “NRG1alpha” or “NRGIA” refers to a soluble form of Pro-neuregulin-1, membrane-bound isoform HRG-alpha proprotein having the GenBank Accession of NCBI NP_039258.1.

As used herein “NRG1beta” or “NRG1B” refers to a soluble form of neuregulin 1 type IV beta 1a having the GenBank Accession ADN85612.1.

As used herein “BTC” refers to a soluble form of Betacellulin, e.g., AAB25452.1.

According to a specific embodiment, the NRG-1A, and NRG-1B comprise less than 90 amino acids which constitute at least a portion of the EGF-like domain of the ligand and excludes the Ig domain. According to a specific embodiment, the ligand portion is 40-100, 40-90, 45-100, 40-60, 45-90, 50-100, 55-90, 52-85, 45-90 amino acids in length.

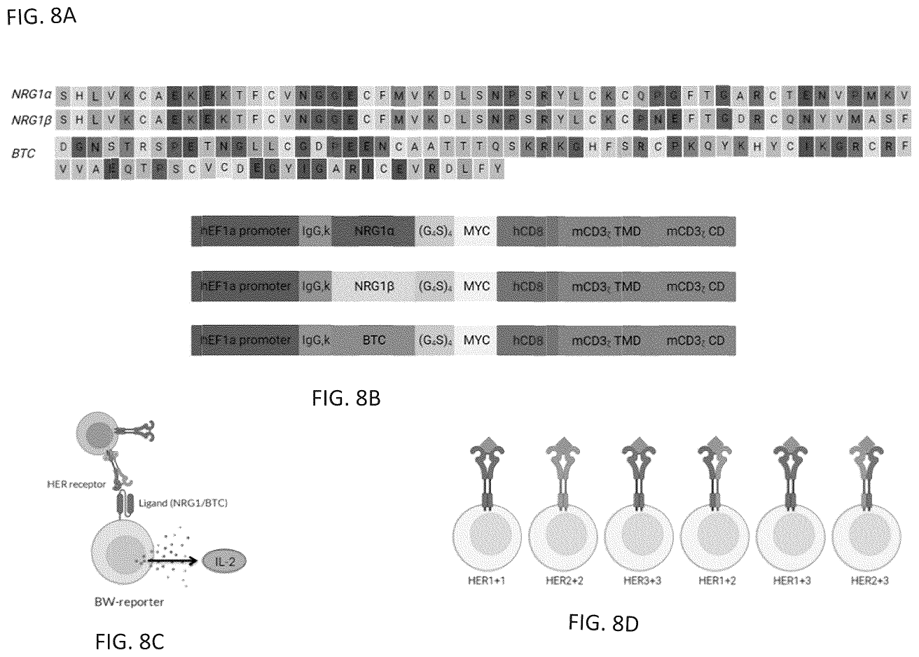

According to a specific embodiment, the NRG-1A is as set forth in SEQ ID NO: 50 of FIG. 8A. Core domain taken from NCBI NP_039258.1 is 177−229=53 AA. It will be appreciated that amino acids coordinates 177 to 181 (5 AA) are not considered as part of the EGF-like protein and thus are from a disordered domain.

```
    1 MSERKEGRGK GKGKKKERGS GKKPESAAGS QSPALPPRLK EMKSQESAAG SKLVLRCETS
  61 SEYSSLRFKW FKNGNELNRK NKPQNIKIQK KPGKSELRIN KASLADSGEY MCKVISKLGN
121 DSASANITIV ESNEIITGMP ASTEGAYVSS ESPIRISVST EGANTSSSTS TSTTGTSHLV
181 KCAEKEKTFC VNGGECFMVK DLSNPSRYLC KCQPGFTGAR CTENVPMKVQ NQEKAEELYQ
241 KRVLTITGIC IALLVVGIMC VVAYCKTKKQ RKKLHDRLRQ SLRSERNNMM NIANGPHHPN
301 PPPENVQLVN QYVSKNVISS EHIVEREAET SFSTSHYTST AHHSTTVTQT PSHSWSNGHT
361 ESILSESHSV IVMSSVENSR HSSPTGGPRG RLNGTGGPRE CNSFLRHARE TPDSYRDSPH
421 SERYVSAMTT PARMSPVDFH TPSSPKSPPS EMSPPVSSMT VSMPSMAVSP FMEEERPLLL
481 VTPPRLREKK FDHHPQQFSS FHHNPAHDSN SLPASPLRIV EDEEYETTQE YEPAQEPVKK
541 LANSRRAKRT KPNGHIANRL EVDSNTSSQS SNSESETEDE RVGEDTPFLG IQNPLAASLE
601 ATPAFRLADS RTNPAGREST QEEIQARLSS VIANQDPIAV
(Whole sequence is SEQ ID NO: 53 and the NRG-1A core
used in the chimeric protein is SEQ ID NO: 50).
```

According to a specific embodiment, the NRG-1B is as set forth in SEQ ID NO: 51 of FIG. 8A. Core domain taken from ADN85612.1 is 122−174=53 AA.

```
    1 mgkgragrvg ttalpprlke mksqesaags klvlrcetss eysslrfkwf kngnelnrkn
  61 kpqnikiqkk pgkselrink asladsgeym ckvisklgnd sasanitive snatststtg
121 tshlvkcaek ektfcvngge cfmvkdlsnp srylckcpne ftgdrcqnyv masfykhlgi
181 efmeaeelyq krvltitgic iallvvgimc vvaycktkkq rkklhdrlrq slrsernnmm
241 niangphhpn pppenvqlvn qyvsknviss ehivereaet sfstshytst ahhsttvtqt
301 pshswsnght esilseshsv ivmssvensr hssptggprg ringtggpre cnsflrhare
361 tpdsyrdsph seryvsamtt parmspvdfh tpsspkspps emsppvssmt vsmpsmavsp
421 fmeeerplll vtpprlrekk fdhhpqqfss fhhnpahdsn slpasplriv edeeyettqe
481 yepagepvkk lansrrakrt kpnghvanrl evdsntssqs snsesetede rvgedtpflg
541 iqnplaasle atpafrlads rtnpagrfst qeeiqarlss vianqdpiav
(Whole sequence is SEQ ID NO: 54 and the NRG-1B core
used in the chimeric protein is SEQ ID NO: 51).
```

According to a specific embodiment, the BTC is as set forth in SEQ ID NO: 52 of FIG. 8A. Core domain taken from AAB25452.1 32−111=80 AA.

```
    1 mdraarcsga sslplllala lglvilhcvv
        adgnstrspe tngllcgdpe encaatttas
  61 krkghfsrcp kqykhycikg rerfvvaeqt
        pscvcdegyi garcervdlf ylrgdrgqil
121 vicliavmvv fiilvigvct cchplrkrrk
        rkkkeeemet lgkditpine dieetnia
(Whole sequence is SEQ ID NO: 55 and
the BTC core used in the chimeric
protein is SEQ ID NO: 52).
```

According to a specific embodiment, the cancer-associated receptor is vascular endothelial growth factor receptor.

According to a specific embodiment, the ligand is VEGFA.

According to a specific embodiment, the ligand/signaling sequences used in the chimeric polypeptide are of mammalian origin, e.g., human.

Homologs of any of the contemplated sequences here are also included under the scope of the present invention according to some embodiments.

Thus, according to a specific embodiment, the amino acid sequence of the ligand is a fragment (e.g., comprising the receptor binding domain and optionally the dimerization inducing site) or a homolog of the ligand molecule, also referred to herein as functional equivalent, as long as it is capable of binding the receptor.

According to a specific embodiment, the ligand is not a recombinant antibody, or specifically a fragment thereof, e.g., scFv.

Typically, the ligand is a membrane anchored ligand in its native form, though it is processed for secretion.

According to a specific embodiment, the active domain of the ligand is devoid of the native transmembrane domain and cytoplasmic domain, which is replaced by that of the cell signaling module. According to a specific embodiment, the amino acid sequence of the ligand comprises the extracellular domain which mediates receptor binding. According to other embodiments, the active domain of the ligand is followed by at least one of a native transmembrane domain and cytoplasmic domain, while a heterologous signaling module is further attached.

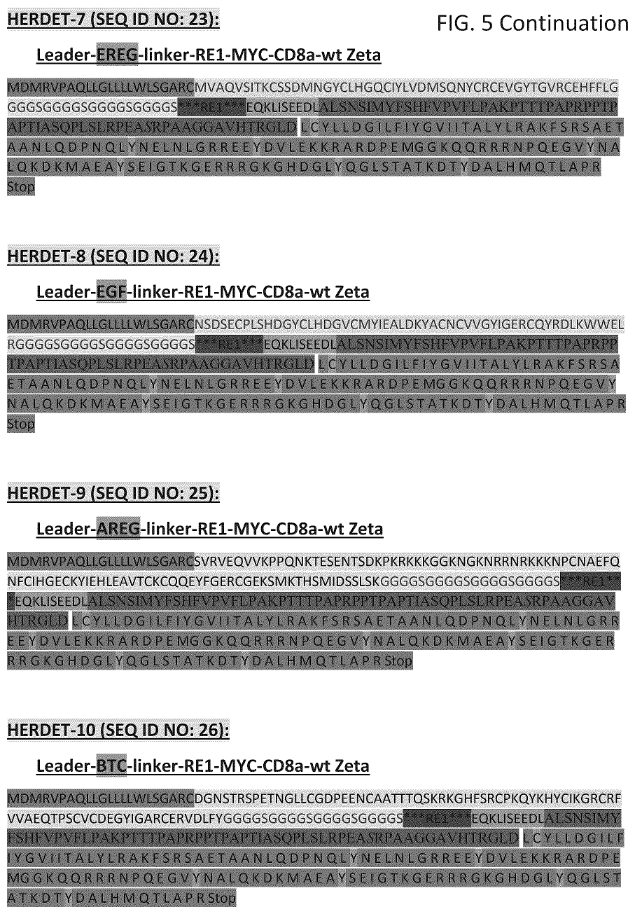

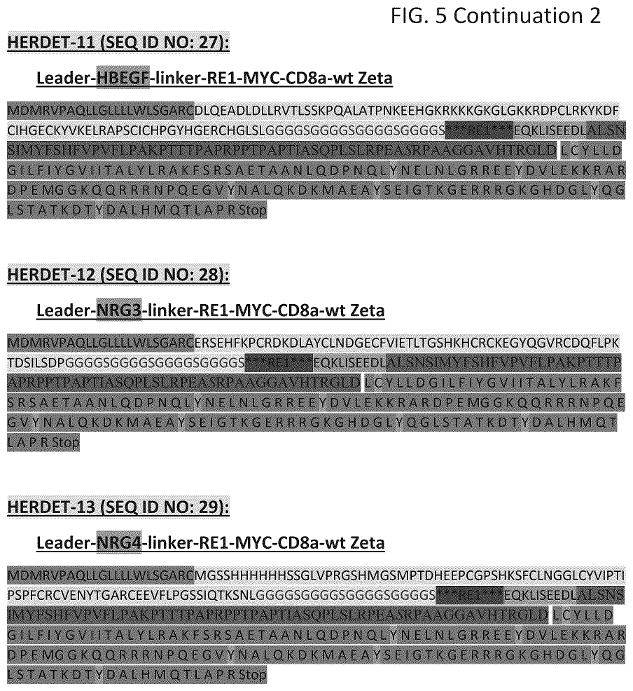

FIG. 5 shows some examples of ligand sequences which can be used in accordance to some embodiments of the invention.

Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the native sequence, as long as the activity e.g., receptor binding and dimerization is retained.

According to some embodiments, the homolog is at least 60%, 70%, 75%, 80%, 85%, 90%, 91%, 92%, 93%, 94%, 95%, 96%, 97%, 98%, 99% identical to the native ligand sequence as long as binding of the ligand to the cognate receptor is maintained (at least 90% affinity to the cognate receptor as compared to the native ligand sequence, e.g., comprising SEQ ID Nos. 17, 18 or 26)

As mentioned, neither the receptor nor the ligand is an immune checkpoint molecule.

As used herein “an immune checkpoint molecule” refers to at least the portion of an immune checkpoint molecule that is capable of binding a ligand thereof which modulates its activity. It is typically an immune checkpoint receptor. These immune checkpoint molecules are regulatory molecules that maintain immune homeostasis in physiological conditions. By sending T cells a series of co-stimulatory or co-inhibitory signals via receptors, immune checkpoints can both protect healthy tissues from adaptive immune response and activate lymphocytes to remove pathogens effectively. However, due to their mode of action, suppressive immune checkpoints serve as unwanted protection for cancer cells.

Examples of immune checkpoint molecules and their ligands that are excluded according to some embodiments of the present invention are selected from the group consisting of CTLA4, PD-1. LAG3, TIGIT, TIM3, VISTA, CEACAM1, CD28, OX40, CD137 (4-1BB), GITR, ICOS, CD27, CD80, CD86, PD-L1, PD-L2, MHC class II/lectins, CD155, Galectin 9, VSIG-3, B7, CD80, CD86, OX40L, CD137L, GITRL, ICOSLG and CD70.

As used herein a “cell signaling module” refers to a portion of a signaling molecule that elicits signal transduction in a direct or indirect response to an extracellular signal, e.g., ligand binding.

“Activation” or “activated” in the context of signaling can be dimerization, protein-protein interaction, phosphorylation, de-phosphorylation, post-translational modification, migration, mobilization, combination of any of the foregoing or the like.

According to some embodiments, the portion is of a cell membrane receptor or cell membrane adapter associated with a signaling capacity that elicits signal transduction in a direct or indirect response to an extracellular signal.

Typically, the cell signaling module is of a cell surface receptor or associated with a cell-surface receptor e.g., T cell receptor complex. T cells co-stimulatory receptor, B-cell receptor complex, G protein-coupled receptor, cytokine receptors, growth factor receptor, tyrosine or Ser/Thr-specific receptor-protein kinase, integrin, Toll-like receptor, ligand gated ion channels or enzyme-linked receptors.

For example, the intracellular portion are of an enzyme-linked receptor. Various classes of enzyme-linked receptors are known and each of which is contemplated according to some embodiments of the invention. For example, receptor tyrosine kinase that phosphorylate specific tyrosines of intracellular signaling proteins; Tyrosine-kinase-associated receptors that associate with intracellular proteins that have tyrosine kinase activity; Receptor-like tyrosine phosphatases that remove phosphate groups from tyrosines of specific intracellular signaling proteins. Receptor serine/threonine kinases that phosphorylate specific serines or threonines on associated latent gene regulatory proteins; Receptor guanylyl cyclases that directly catalyze the production of cyclic GMP in the cytosol; and Histidine-kinase-associated receptors activate a “two-component” signaling pathway in which the kinase phosphorylates itself on histidine and then immediately transfers the phosphate to a second intracellular signaling protein.

The binding of an extracellular signal (and in this case, receptor) typically changes the orientation of transmembranal structures, in some cases forming a dimer or a higher oligomer. In other cases the oligomirezation (on the cancer cell) occurs before ligand binding and the ligand causes a reorientation of the receptor chains in the membrane. In either case, the rearrangement induced in cytoplasmic tails of the chimeric ligands initiates an intracellular signaling process.

Autophosphorylation of the cytoplasmic tail of receptor tyrosine kinases contributes to the activation process in two ways. First, phosphorylation of tyrosines within the kinase domain increases the kinase activity of the enzyme. Second, phosphorylation of tyrosines outside the kinase domain creates high-affinity docking sites for the binding of a number of intracellular signaling proteins in the target cell. Each type of signaling protein binds to a different phosphorylated site on the activated receptor because it contains a specific phosphotyrosine-binding domain that recognizes surrounding features of the polypeptide chain in addition to the phosphotyrosine. Once bound to the activated kinase, the signaling protein may itself become phosphorylated on tyrosines and thereby activated; alternatively, the binding alone may be sufficient to activate the docked signaling protein.

Alternatively, the signaling module is of a tyrosine phosphatase that acts as a cell surface receptor. Some comprise an SH2 domain and thus are called SHP-1 and SHP-2, additional compositions of signaling modules are described in the following references: SynNotch approach—cell 164, 1-10, Feb. 11, 2016 Protein-Logic based on HER2 and EGFR (M. J. Lajoie et al, Science 10.1126/science.aba6527 (2020), SUPRA-CAR technology (zipper TECHNOLOGY), Cell 173, May 31, 2018, incorporated herein by reference.

According to a specific embodiment, the cell signaling module is absent or inactive, or suppressed in the absence of stimulation or activation in the reporter cell, as describe herein below in more details.

According to a specific embodiment the cell signaling module comprises a transmembrane domain and/or a cytoplasmic portion of a cell signaling receptor.

According to a specific embodiment the cell signaling module comprises a transmembrane domain and/or a cytoplasmic portion of a receptor kinase.

According to a specific embodiment the receptor kinase is a tyrosine kinase or serine/threonine kinase.

According to a specific embodiment the cell signaling module comprises an adaptor molecule.

According to a specific embodiment the cell signaling module comprises a CD3 zeta chain (e.g., see Examples 1 and 2).

According to a specific embodiment the activation of the cell signaling module is by dimerization, oligomerization and/or post-translational modification.

According to a specific embodiment, the cell signaling module is selected of a species of the host cell such that it can mediate a signaling cascade in an operable manner. According to a specific embodiment, the signaling module and optionally the transmembrane domain is non-human, e.g., murine.

According to a specific embodiment, the ligand (extracellular) is typically N-terminus to the cell signaling module (intracellular).

As used herein, the term “polypeptide” or “peptide” encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells.

The term “amino acid” or “amino acids” typically refers to amino acids which can be used in recombinant protein synthesis.

When referring to “an amino acid sequence” the meaning is to the chemical embodiment of the term and not the literal embodiment of the term.

Alternatively or additionally, the polypeptides of some embodiments of the invention may be synthesized by any techniques that are known to those skilled in the art of peptide synthesis, such as, but not limited to, recombinant techniques.

Large scale peptide synthesis is described by Andersson Biopolymers 2000; 55(3):227-50.

To express the chimeric polypeptide, the polynucleotide is cloned into a nucleic acid expression construct and introduced into a cell, i.e., a reporter cell.

Thus to express exogenous polynucleotides in cells, a polynucleotide sequence encoding the chimeric polypeptide is preferably ligated into a nucleic acid construct suitable for cell expression. Such a nucleic acid construct includes a promoter sequence for directing transcription of the polynucleotide sequence in the cell in a constitutive or inducible manner.

As mentioned, the nucleic acid construct of some embodiments of the invention can also utilize nucleic acid homologues which exhibit the desired activity (e.g., receptor binding). Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the native sequences, as determined using the BestFit software of the Wisconsin sequence analysis package, utilizing the Smith and Waterman algorithm, where gap weight equals 50, length weight equals 3, average match equals 10 and average mismatch equals-9.

Constitutive promoters suitable for use with some embodiments of the invention are promoter sequences which are active under most environmental conditions and most types of cells such as the cytomegalovirus (CMV) and Rous sarcoma virus (RSV).

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for membrane presentation. Preferably the signal sequence for this purpose is a mammalian signal sequence or the signal sequence of the polypeptide variants of some embodiments of the invention. Examples are provided hereinbelow in the Examples section.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide. This is of specific significance when a plurality of repoter chimeric polypeptides are included in a single vector (e.g., et least 2 of Herdet1, Herdet2, Herdet3 and Herdet4).

It will be appreciated that the individual elements comprised in the expression vector can be arranged in a variety of configurations. For example, enhancer elements, promoters and the like, and even the polynucleotide sequence(s) encoding the polypeptide can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein.

Introduction of nucleic acids by viral infection offers several advantages over other methods such as lipofection and electroporation, since higher transfection efficiency can be obtained due to the infectious nature of viruses.

According to a specific embodiment, the vector is a Lentiviral vector (e.g., pHAGE2).

Other than containing the necessary elements for the transcription and translation of the inserted coding sequence, the expression construct of some embodiments of the invention can also include sequences engineered to enhance stability, production, purification, yield or toxicity of the expressed peptide.

Mammalian cells and especially human cells are preferably used to express the polypeptides of some embodiments of the invention.

Thus, the reporter cell can also be referred to as a transgenic cell.

The polynucleotide of some embodiments of the invention can be introduced into cells by any one of a variety of known methods within the art. Such methods can be found generally described in Sambrook et al., [Molecular Cloning: A Laboratory Manual, Cold Springs Harbor Laboratory, New York (1989, 1992)]; Ausubel et al., [Current Protocols in Molecular Biology, John Wiley and Sons, Baltimore, Maryland (1989)]; Chang et al., [Somatic Gene Therapy, CRC Press, Ann Arbor, MI (1995)]; Vega et al., [Gene Targeting, CRC Press, Ann Arbor MI (1995)]; Vectors [A Survey of Molecular Cloning Vectors and Their Uses, Butterworths, Boston MA (1988)] and Gilboa et al. [Biotechniques 4 (6): 504-512 (1986)] and include, for example, stable or transient transfection, lipofection, electroporation and infection with recombinant viral vectors. Introduction of the polynucleotide can be in a stable or transient manner.

The “reporter cell” is any cell which can be used as a host cell for recombinant expression of the polynucleotide and in which the cell signaling module is capable of eliciting signaling.

According to some embodiments, the reporter cell can be a cell line or a primary cell.

According to some embodiments, the reporter cell is typically isolated and does not form a part of a tissue.

According to some embodiments, the reporter cell is an immune cell, e.g., T lymphocyte, B lymphocyte and the like.

According to a specific embodiment, the cell is a mammalian cell, e.g., human or murine cell.

According to a specific embodiment, the immune cell is an antigen presenting cell.

According to a specific embodiment, the immune cell is not an antigen presenting cell.

According to a specific embodiment, the immune cell line is a mouse BW5147 thymoma cell (ATCC TIB-47™).

According to some embodiments, the reporter cell is a non-immune cell which is typically used for recombinant expression, e.g., CHO, 293T, NIH3T3, COS7 and the like.

In some embodiments, a cell in which the assayed ligand(s)/receptor(s) are not natively expressed is preferably used.

Activation of the signaling module can be done by detecting induction (e.g., expression) of a reporting molecule (e.g., IL-2, IL-8) or a fluorescent or bioluminescent signal, for instance using a promoter responsive element(s), responding at the end of the signaling module cascade, linked to a nucleic acid sequence encoding a bioluminescent or fluorescent molecule.

According to a specific embodiment, the reporter gene encodes an enzyme whose catalytic activity can be detected by a simple assay method or a protein with a property such as intrinsic fluorescence or luminescence so that expression of the reporter gene can be detected in a simple and rapid assay requiring minimal sample preparation. Non-limiting examples of enzymes whose catalytic activity can be detected are Luciferase, beta Galactosidase, Alkaline Phosphatase.

The term “protein with intrinsic fluorescence” refers to a protein capable of forming a highly fluorescent, intrinsic chromophore either through the cyclization and oxidation of internal amino acids within the protein or via the enzymatic addition of a fluorescent co-factor. The term “protein with intrinsic fluorescence” includes wild-type fluorescent proteins and mutants that exhibit altered spectral or physical properties. The term does not include proteins that exhibit weak fluorescence by virtue only of the fluorescence contribution of non-modified tyrosine, tryptophan, histidine and phenylalanine groups within the protein. Proteins with intrinsic fluorescence are known in the art, e.g., green fluorescent protein (GFP)), red fluorescent protein (RFP), Blue fluorescent protein (BFP, Heim et al. 1994, 1996), a cyan fluorescent variant known as CFP (Heim et al. 1996; Tsien 1998); a yellow fluorescent variant known as YFP (Ormo et al. 1996; Wachter et al. 1998); a violet-excitable green fluorescent variant known as Sapphire (Tsien 1998; Zapata-Hommer et al. 2003); and a cyan-excitable green fluorescing variant known as enhanced green fluorescent protein or EGFP (Yang et al. 1996) and can be measured e.g., by live cell imaging (e.g., Incucyte) or fluorescent spectrophotometry. “Reduced binding” refers to a decrease in affinity for the respective interaction, as measured for example by SPR. For clarity the term includes also reduction of the affinity to zero (or below the detection limit of the analytic method), i.e. complete abolishment of the interaction.

The method can use more than one reporter e.g., a first reporter and a second reporter, which are different in the signal they produce. The second reporter can be used to detect an organelle for instance, such as to mark a cell membrane, a cell nucleus, a cell cytoplasm and the like. The second reporter can be also a chemical dye i.e., non-proteinaceous.

According to a specific embodiment, the first reporter and optionally second reporter are fluorescent or bioluminescent.

Alternatively or additionally, determining activation is by analyzing a phenotype selected from the group consisting of cell proliferation, death, arrest, migration, morphology, cell localization in a tissue, receptor ligand interactions and the like.

Methods of analyzing interleukin in culture are well known in the art and some are based on commercially available kits.

According to some embodiments, the method further comprises determining activation of the cell signaling module in the reporter cell in the presence of soluble ligand expressed by the reporter cell, to determine background activation in case a non null system is used.

“A null system” refers to cells in which the assayed ligand-receptor pair is not naturally expressed or expressed to a level which is not detectable by protein expression or activity assays, such as Western, ELISA and the like.

In an alternative embodiment, the reporter cell is of a different species than that of the chimeric protein (e.g., animal (e.g., mouse) vs. human).

It will be appreciated that due to the high sensitivity of the cells, the methods described herein can be employed using as little as 10² cells or at least 10² cells (e.g., 10²-10⁴, 10²-10³, 10², 100×10³).

The reporter cells described herein can be used in methods which qualify/quantify receptors on cancer cells.

Thus, according to an aspect of the invention, there is provided a method of detecting presence and/or activity of a cancer-associated receptor in a cancer cell, the method comprising:
(a) contacting the cancer cell with the reporter cell as described herein;
(b) determining activation of said cell signaling module in the reporter cell, said activation being indicative of the presence and/or activity of the cancer-associated receptor or dimers or heterodimers thereof in the cancer cell.

As used herein, the term “cancer” encompasses both malignant and pre-malignant cancers.

Cancers which can be analyzed and eventually treated by the methods of some embodiments of the invention can be any solid or non-solid cancer and/or cancer metastasis. According to a specific embodiment, the cancer is a solid tumor.

Examples of cancer include but are not limited to, carcinoma, lymphoma, blastoma, sarcoma, and leukemia. More particular examples of such cancers include squamous cell cancer, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; Burkitt lymphoma, Diffused large B cell lymphoma (DLBCL), high grade lymphoblastic NHL; high-grade small non-cleaved cell NHL; bulky disease NHL; mantle cell lymphoma; AIDS-related lymphoma; and Waldenstrom's Macroglobulinemia); T cell lymphoma, Hodgkin lymphoma, chronic lymphocytic leukemia (CLL); acute lymphoblastic leukemia (ALL); Acute myeloid leukemia (AML), Acute promyelocytic leukemia (APL), Hairy cell leukemia; chronic myeloblastic leukemia (CML); and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amenable for treatment of the invention also include metastatic cancers.

According to specific embodiments, the cancer comprises pre-malignant cancer.

Pre-malignant cancers (or pre-cancers) are well characterized and known in the art (refer, for example, to Berman J J. and Henson D E., 2003. Classifying the precancers: a metadata approach. BMC Med Inform Decis Mak. 3:8). Classes of pre-malignant cancers amenable to treatment via the method of the invention include acquired small or microscopic pre-malignant cancers, acquired large lesions with nuclear atypia, precursor lesions occurring with inherited hyperplastic syndromes that progress to cancer, and acquired diffuse hyperplasias and diffuse metaplasias. Examples of small or microscopic pre-malignant cancers include HGSIL (High grade squamous intraepithelial lesion of uterine cervix), AlN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Examples of precursor lesions occurring with inherited hyperplastic syndromes that progress to cancer include atypical mole syndrome, C cell adenomatosis and MEA. Examples of acquired diffuse hyperplasias and diffuse metaplasias include AIDS, atypical lymphoid hyperplasia, Paget's disease of bone, post-transplant lymphoproliferative disease and ulcerative colitis.

According to specific embodiments, the cancer is Acute Lymphocytic Leukemia (ALL), Acute Myeloid Leukemia, Anal Cancer, Basal Cell Carcinoma, B-Cell Non-Hodgkin Lymphoma, Bile Duct Cancer, Bladder Cancer, Breast Cancer, Cervical Cancer, Chronic Lymphocytic Leukemia (CLL), Chronic Myelocytic Leukemia (CML), Colorectal Cancer, Cutaneous T-Cell Lymphoma, Diffuse Large B-Cell Lymphoma, Endometrial Cancer, Esophageal Cancer, Fallopian Tube Cancer, Follicular Lymphoma, Gastric Cancer, Gastroesophageal (GE) Junction Carcinomas, Germ Cell Tumors, Germinomatous (Seminomatous), Germ Cell Tumors, Glioblastoma Multiforme (GBM), Gliosarcoma, Head And Neck Cancer, Hepatocellular Carcinoma, Hodgkin Lymphoma, Hypopharyngeal Cancer, Laryngeal Cancer, Leiomyosarcoma, Mantle Cell Lymphoma, Melanoma, Merkel Cell Carcinoma, Multiple Myeloma, Neuroendocrine Tumors, Non-Hodgkin Lymphoma, Non-Small Cell Lung Cancer, Oral Cavity (Mouth) Cancer, Oropharyngeal Cancer, Osteosarcoma, Ovarian Cancer, Pancreatic Cancer, Peripheral Nerve Sheath Tumor (Neurofibrosarcoma), Peripheral T-Cell Lymphomas (PTCL), Peritoneal Cancer, Prostate Cancer, Renal Cell Carcinoma, Salivary Gland Cancer, Skin Cancer, Small-Cell Lung Cancer, Soft Tissue Sarcoma, Squamous Cell Carcinoma, Synovial Sarcoma, Testicular Cancer, Thymic Carcinoma, Thyroid Cancer, Ureter Cancer, Urethral Cancer, Uterine Cancer, Vaginal Cancer or Vulvar Cancer.

According to specific embodiments, the cancer is Acute myeloid leukemia, Bladder Cancer, Breast Cancer, chronic lymphocytic leukemia, Chronic myelogenous leukemia, Colorectal cancer, Diffuse large B-cell lymphoma, Epithelial Ovarian Cancer, Epithelial Tumor, Fallopian Tube Cancer, Follicular Lymphoma, Glioblastoma multiform, Hepatocellular carcinoma, Head and Neck Cancer, Leukemia, Lymphoma, Mantle Cell Lymphoma, Melanoma, Mesothelioma, Multiple Myeloma, Nasopharyngeal Cancer, Non Hodgkin lymphoma, Non-small-cell lung carcinoma, Ovarian Cancer, Prostate Cancer or Renal cell carcinoma.

According to specific embodiments, the cancer is selected from the group consisting of Acute Lymphocytic Leukemia (ALL), Bladder Cancer, Breast Cancer, Colorectal Cancer, Head and Neck Cancer, Hepatocellular Carcinoma, Melanoma, Multiple Myeloma, Non-Small Cell Lung Cancer, Non-Hodgkin Lymphoma, Ovarian Cancer, Renal Cell Carcinoma.

According to specific embodiments, the cancer is selected from the group consisting of Gastrointestinal (GI) cancers, Breast Cancer, Ovarian Cancer and Pancreatic Cancer.

The cancer cell can be a primary cell taken from a tissue biopsy or a cell line.

According to a specific embodiment, the cancer cell is comprised in a tissue biopsy.

According to a specific embodiment, the cancer cells is in a biological sample selected from the group consisting of an FFPE blocked, Patient-Derived Xenografts (PDXs) and Cell line Derived Xenografts (CDXs)

According to some embodiments, the tissue biopsy is fresh, not subjected to any preservation protocol. i.e., fixation protocol.

According to other embodiments, the tissue biopsy has been subject to fixation.

According to some embodiments, the tissue biopsy is subjected to antigen retrieval.

For example, when the tissue biopsy has been preserved with formaldehyde, a highly reactive compound, it may a variety of chemical modifications that can reduce the detectability of proteins in biomedical procedures. Antigen retrieval is an approach to reducing or eliminating these chemical modifications. The two primary methods of antigen retrieval are heat-mediated epitope retrieval (HIER) and proteolytic induced epitope retrieval (PIER).

Thus, contacting with the reporter cell is preferably and according to some embodiments of the invention done following antigen retrieval.

The cancer cell can be used following freezing/thawing or immediately upon biopsy retrieval.

According to a specific embodiment, the cancer cell is a primary cell.

Contacting can be effected in a culture dish such as in a petri dish or flask, or in a multiwall configuration e.g., 96 or more wells, when a plurality of ligands are assayed and/or a plurality of inhibitors.

According to some embodiment, the contacting is effected such that the tumor tissue or cells derived therefrom is/are seeded on the plate and the reporter cells are seeded thereon.

Contacting can be effected in the presence and/or absence of an inhibitor of the cancer-associated receptor.

According to some embodiments of the invention, the inhibitor is selected from the group consisting of an antibody, an aptamer and a peptide.

Following is a non-limiting list of inhibitors which can be used in accordance with some embodiments of the invention.

TABLE 4
Some mAbs against GFRs for treating cancer (adapted from Tiash
et al. J Cancer Metastasis Treat 2015; 1: 190-200).

| | | | | | Resistance | Status |
|---|---|---|---|---|---|---|
| | | Targeted | | Mechanism of | (known | (highest |
| Name of mAB | Target | stages | Indication | action | mechanism) | level) |
| Cetuximab | ErbB1 | Suppresses | Metastatic | Inhibition | Yes (mutations | Approved |
| (human IgG1) | | cell growth | colorectal; | EGFR | in a number of | |
| (use as single or | | and | head and neck | signaling | diverse genes | |
| conjunction with | | metastasis | carcinoma | (down | are blamable | |
| radiotherapy) | | | | regulates | for intrinsic | |
| | | | | active | resistance) | |
| | | | | EGFRVII) | | |
| | | | | and ADCC | | |
| Panitumumab | ErbB1 | Suppresses | Metastatic | Prevents | | Approved |
| (human IgG2) | | cell growth | colorectal | EGFR | | |
| (use as single | | | cancer | activation | | |
| agent) | | | | | | |
| Trastuzumab | ErbB2 | Suppresses | HER2+ | Inhibition | Yes | Approved |
| (humanized IgG1) | | cell growth | metastatic | ErbB2 | (overexpression | |
| (use as single or | | and | breast cancer | signaling and | of membrane | |
| as adjuvant for | | angiogenesis, | | ADCC | associated | |
| chemotherapy) | | induces cell | | | glycoprotein | |
| | | death | | | MUC4; | |
| | | | | | increase IGF1R | |
| | | | | | signaling are | |
| | | | | | some of the | |
| | | | | | reasons that | |
| | | | | | confer | |
| | | | | | resistance) | |
| Ganitumab | IGF1R | Inhibits cell | Non-Hodgkin | Blocks IGF-1 | Yes (calcium | Clinical |
| (human IgG1) | | growth, | lymphoma, | and-2 binding | dependent | trials |
| (use as single or | | delays tumor | metastatic | to IGF1R | proliferation | (passed |
| combined with | | progression | pancreatic | without | effects acquire | phase II) |
| different | | | cancer, | crosslinking | resistance in | |
| neoplastic drugs) | | | metastatic | with IR, | prostate cancer | |
| | | | Ewing family | inhibits | cells) | |
| | | | of tumors | activation of | | |
| | | | | IGF1R | | |
| | | | | homodimer | | |
| | | | | and IGF1R/IR | | |
| | | | | heterodimer | | |
| Cixutumumab | IGF1R | Induces | Solid tumors, | Prevents | NA | Clinical |
| (human IgG1) | | cancer cell | Ewing | IGF1 binding | | trials |
| (use as single or | | apoptosis, | sarcoma | to receptor | | (phase I- |
| combined with | | decreases | family tumors | and | | II) |
| different | | cell | | subsequent | | |
| neoplastic drugs) | | proliferation | | activation of | | |
| | | | | PI3K/AKT | | |
| | | | | survival | | |
| | | | | pathway, | | |
| | | | | mediates | | |
| | | | | receptor | | |
| | | | | internalization | | |
| | | | | and | | |
| | | | | degradation | | |
| PF03446962/Anti- | TGF-βR | Prevents | Transitional | Disrupts co- | NA | Clinical |
| Alk1 (human) | | angiogenesis | cell carcinoma | localization of | | trials |
| | | (dose | of bladder | endothelial | | (phase II) |
| | | dependent) | | cells with | | |
| | | | | perivascular | | |
| | | | | cells and | | |
| | | | | reduces blood | | |
| | | | | flow | | |
| Ramucirumab | VEGFR2 | Inhibits | Hepatocellular, | Blocks VEGF | Yes (VEGF- | Clinical |
| (human IgG1): | | tumor | renal cell, and | binding to the | axis dependent | trials |
| use as single or | | angiogenesis | ovarian | receptor and | pathway is | (phase II) |
| with neoplastic | | and growth | carcinomas | thus VEGF- | involved for | |
| drugs | | | | signaling and | resistance) | |
| | | | | subsequently | | |
| | | | | angiogenesis | | |
| 1B3 (used in | Mouse | Inhibits | Pancreatic and | Blocks | NA | Preclinical |
| combination with | PDGFRβ | angiogenesis | a nonsmall cell | PDGFR | | trial |
| mAB against | | | lung tumor | binding with | | |
| antitumor/anti- | | | xenograft | receptor, | | |
| angiogenic agent) | | | models | ligand- | | |
| IMC-2C5 | Both | Delays | | stimulated | NA | Preclinical |
| (human) | mouse | growth (cell | | activation of | | tr |
| | and | specific), | | PDGFRβ and | | |
| | human | inhibits | | downstream | | |
| | PDGFRβ | angiogenesis | | signaling | | |
| | | | | molecules in | | |
| | | | | tumor cells | | |

Contacting can be effected first, between the cancer cell and the reporter cell and then subjecting to the inhibitor.

Alternatively, contacting can be effected in the presence of the inhibitor (simultaneous incubation).

Activation of the cell signaling module is determined using methods known in the art and available kits.

Typically, activation is determined relative to a control, such as a negative control to determine base activation of the cell signaling module.

According to some embodiments, the negative control is under the same conditions yet in the absence of the inhibitor or with isotype matched control. Alternatively using normal cells which are adjacent to the tumor (e.g., on the same tissue sample). Such a control can also be used to determine treatment toxicity to normal tissues.

Standards can be used to calibrate the signal. One can prepare a calibration curve based on cells which express known levels of the cognate receptors and their response to the reporter cells is determined in order to decipher the level of the receptors, dimers or heterodimers of same.

Activation of the cell signaling module is indicative of the presence and/or activity of the receptor in the cancer cell and the effect of the inhibitor.

It will be appreciated that effect of the inhibitor on the activation is indicative of the specificity of activation of the cell signaling module.

The level of activation can be calculated using various algorithms including those which employ scoring. In such a case, the scoring of the response may be based on a scoring combination of (a) the level of activation without the inhibitor (i.e., maximal activation of the reporter cell); and (b) the fraction of reduction of activation after adding the inhibitor.

According to some embodiments, the quantification of the receptor is done without immunohistochemistry (IHC).

According to some embodiments, the quantification of receptor is corroborated by immunohistochemistry (IHC).

According to some embodiments, the quantification of the receptor is corroborated by transcriptome analysis.

These teachings can be harnessed towards selecting treatments for cancer patients.

Thus, according to an aspect of the invention there is provided a method of treating a subject diagnosed with cancer, the method comprising:
(a) detecting presence and/or activity of a cancer-associated receptor in a cancer cell of the subject as described herein; and
(b) treating the subject with an inhibitor of the receptor when presence or a predetermined threshold of activity of said cancer-associated receptor or dimers or heterodimers thereof is indicated or with another treatment modality when it is not indicated or absent.

Alternatively or additionally, there is provided a method of selecting treatment for a subject diagnosed with cancer, the method comprising:
(a) detecting presence and/or activity of a cancer-associated receptor in a cancer cell of the subject as described herein; and
(b) selecting treatment for the subject with an inhibitor of the receptor when presence or a predetermined threshold of activity of said cancer-associated receptor or dimers or heterodimers thereof is indicated or with another treatment modality when it is not indicated or absent.

According to some embodiments of the invention, the inhibitor is a chimeric antigen receptor (CAR).

According to some embodiments of the invention, the CAR is an effector immune cell expressing the polynucleotide encoding a chimeric polypeptide as described herein.

Methods of designing and synthesizing CARs are well known in the art. The cells can be any effector cell such as a T cell or an NK cell. For further description see Ramirez-Chacon (2022) Ligand-based CART cell: Different strategies to drive T cells in future new treatments. Front. Immunol. 13:932559, which is hereby incorporated by reference in its entirety.

As used herein “another treatment modality” is typically a treatment which is not specific against the receptor or dimers or heterodimers thereof. For example, other biologics targeting other cancer specific targets, chemotherapy, radiotherapy and the like.

As used herein “predetermined threshold” typically refers to at least above 20%, 30%, 40%, 50%, 70%, 2 fold, 5 fold 10 fold or more increase in activity as compared to a negative control in a statistically significant manner.

It will be appreciated that a scoring system can be employed to elucidate activation above a “predetermined threshold”. Such a scoring system can take into account the difference in activation between the presence and absence of the inhibitor. Additionally the surface of each well covered by the patients-derived tissue is taken into account. Calculation of the covered area (tissue surface) is made by imaging analysis of each individual well.

According to a specific embodiment the scoring system is an IcAR-score, based on:

Calculation of TcAR score—The TcAR score is based on calculation between the maximum signal (PC), and the signal obtained with and without blocking with the inhibitor. Moreover, also taken into account was the area of the tissue (surface) that covers the 96 well plates. To compare between experiments and plates the present inventors have used the PC. PCavg- is pooled of all experiments, PCexp- is the positive control of the specific experiment.

$$TcAR score = \{ ( \frac{AvgIL 2 unblocked}{Log 2 surface} ) - ( \frac{AvgIL 2 blocked}{Log 2 surface} ) \} * \frac{PCAvgIL 2}{PCexpIL 2}$$

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology.

According to a specific embodiment, the cancer cell is autologous to the subject.

According to a specific embodiment, the immune cell is autologous to the subject.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Defining Cancer Cells Heterodimer and Homodimer Status of Growth Factor Receptors that are Associated with Cancerous State

### The ErbB (HER) Receptor Family

### Scientific Background

Breast cancer is the most common type of cancer occurring in women worldwide. The expression of the epidermal growth factor receptors (EGFRs) which are tyrosine kinases (RTKs), regulate the progression of the breast cancer. The ErbB family of proteins contains four receptor tyrosine kinases, structurally related to the epidermal growth factor receptor (EGFR), its first discovered member. In humans, the family includes HER1 (EGFR, ErbB1), HER2 (Neu, ErbB2), HER3 (ErbB3), and HER4 (ErbB4). Overexpression of HER2 is observed in approximately 20-30% of the breast cancer tumors, which leads to a more aggressive disease and a high recurrence rate (Mitri, Constantine and O'Regan 2012). A multitude of EGF family ligands have been identified for the HER family. However, no ligand has been identified for HER2. Although, HER2 is present in an active conformation and can undergo ligand-independent dimerization, HER2 is also the preferred heterodimerization partner for other EGF receptors (Graus-Porta et al., 1997).

Various cancers can express one or more of and up to all HER (ErbB) family members on the membrane of the same cancer cell (Thomas and Weihua 2019, Bunn et al., 2001, Scharpenseel et al., 2019, Zheng et al., 2016, Arienti, Pignatta, and Tesei 2019). According to the available literature, human cancer cell lines express HER2 and at least one additional HER family member(s). As mentioned, when HER2 is expressed with other HER members on target cancer cells, the cells can express on their membrane both HER homodimers and HER2-positive heterodimers (Jones, Akita and Sliwkowski 1999, Muthuswamy et al., 1999, Graus-Porta et al., 1997). Since all the HER receptors depict a favorable heterodimerization towards HER2 in cancer patients, predicting the pattern of HER2 heterodimers versus the HER homodimers can be corelated with the clinical response to various anti-HER treatments.

Chemotherapeutic drugs developed against a specific HER are wildly employed in cancer therapy (Oh and Bang 2020); yet their treatment efficacy could be affected by the balance between the homodimeric and the heterodimeric status of the HER family receptors expressed by the cancer cell (intrinsic or acquired resistance) (Ma et al., 2021). It was suggested that epiregulin, secreted by macrophages in the tumor microenvironment might contribute to gefitinib resistance through EGFR/HER2 heterodimerization (Shiqi Ma). Antibody-based drugs were also developed against the HER family. Trastuzumab (Herceptin), a recombinant humanized monoclonal antibody is a widely used treatment for HER2-positive breast cancer patients. Trastuzumab triggers HER2 internalization and degradation through ubiquitination, through ADCC mechanism, or inhibition of MAPK and PI3K/Akt pathways (Vu and Claret 2012). However, trastuzumab resistance is being observed widely, leading to failure in the therapy (Luque-Cabal et al., 2016).

### Solution

A multiplex reporter system based on the “electronic nose” principals that is able to identify the bioavailability of specific HER family homodimers/heterodimers on membrane of target cancer cells. The basis for the reporter system is the multitude of natural HER ligands having different affinities to the HER1, HER3, HER4 homodimers and to the HER1-HER2, HER2-HER3 and HER2-HER4 homodimers. The active part of these natural HER ligands was introduced into a BW5147 (or “BW”)-based reporter system with chain zeta as the reporter element fused to CD8a hinge followed by a linker and a ligand (C to N). Four ligands were elected: NRG1α, NRG1β, NRG2β and TGFα and their active receptor binding domain was introduced upstream of the linker-encoding nucleotide sequence and of a zeta-based reporting system in BW cells (an ErbB-null system) that upon binding of the ligand (HERDET1, HERDET2, HERDET3, and HERDET4, respectively) will secrete IL-2. FIG. 1A illustrates the 4 HERDET reporters and FIG. 1B depicts the possible response pattern of artificial reporters composed from these 4 ligands, as calculated from the ligands' reported affinities in the literature (Jones, Akita and Sliwkowski 1999).

### Materials and Methods

### Construction of HERDET Reporters

HERDET1 (SEQ ID NO: 17), HERDET2 (SEQ ID NO: 18), HERDET3 (SEQ ID NO: 19), HERDET4 (SEQ ID NO: 20) with MYC-CD8α-WT ZETA in PHAGE2 plasmid backbone in BW reporter cells. The sequences employed are disclosed in FIG. 5.

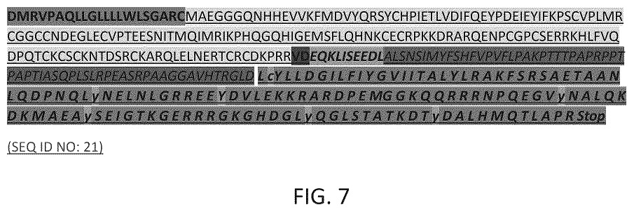

### Explanation for Sequences Detailed in FIG. 5 (and Also in FIG. 7):

Old Green (bold)—Signal peptide of light chain
Yellow (underline)—active part of a Ligand (e.g.—NRG1a, NRG1b, NRG2a, TGFa)
Light Grey (italics)—linker
Strong green (Bold underline)—Restriction Enzyme 1=RE1=SAL-I=DV in AA
Bright green (bold italics)—Myc tag 10AA peptide
Red (italics underline)—hinge (CD8a, mutated for Cyst)
Pink (bold underline italics)—murine Zeta inc. cyst in ectodomain (in bright green (small caps) marked the important AA)

### Screening of JIM T1 (WT and KO for HER2) for HER1, HER2, HER3 Expression

JIMT1 cells natively express high levels of all 4 HERs on their cell surface (Mol Cancer Ther. 2004 December; 3(12):1585-92). Using the CRISPR method the present inventor generated JIMT1-HER2 KO cells (gRNAs are provided in SEQ ID Nos: 40-1, 42-3, 44-5, 46-7, 48-9). In the present system the JIMT1 and JIMT1-HER2 KO were used as target tumor cells for HERDET1 and HERDET2 (as in FIG. 5).

### Culturing of Cells

JIM T1 (WT and KO) were cultured in complete DMEM supplementted with 10% FBS and when confluent, trypsinised. 50,000 cells were taken in 96 U bottom plate and washed once with 100 ul of 1×PAF.

### Staining

50 ul of antibodies (diluted 1:250 in 1×PAF) were added to cells and incubated on ice for 30 min. The cells were washed twice with 1×PAF and 100 ul DAPI (diluted 1:1000 in 1×PAF) were added. FACS analysis followed.

TABLE 5
Antibodies

| Isotype control | Test |
|---|---|
| PE mouse IgG1, k (400114) | PE anti-human EGFR (352903) (HER1) |
| FITC mouse IgG1, k (400110) | FITC anti-human CD340 HER2 (324404) |
| APC anti-mouseCD335 | APC anti-human HER3 (324708) |
| (137607) | |

mIL-2 secretion by HERDET1 and HERDET2 with JIM T1 target cells (WT and KO)

HERDET 1 and HERDET2 were grown in complete RPMI while JIM T1 (WT and KO) cells were cultured in complete DMEM. After trypsinization JIM T1 cells (WT and KO) were suspended in complete RPMI and 100 ul of 25,000 cells were seeded in a 96 well flat bottom plate and incubated for 16-18 h. 100 ul of 50,000 HERDET cells/well were added and incubated for 16-18 h at 37° C. IL-2 secretion was then quantified by performing an ELISA assay, as follows. ELISA plates were coated with purified anti-mouse IL2 (1:500 diluted in Na2HPO4 Coating buffer (0.1M pH=9)) and incubated overnight at 4° C. The plate was washed twice with PBST and blocked with 10% FBS. The plate was incubated for 2 h at room temperature, followed by washing with PBST twice. The experimental plate was centrifuged at 1200 rpm for 5 min, and medium samples were collected and added to the ELISA plate. The plate was incubated for 3 h at room temperature and then washed with PBST 6 times. Biotin anti-mouse IL2 (1:500 diluted in 2% FBS) was added to the plate and incubated for 1 h at room temperature. The plate was washed 6 times with PBST, and SA-HRP (1:750 diluted in 2% FBS) was added. After incubating the plate for 30 min, TMB was added, and the reading was recorded in the spectrophotometer plate reader at 2=650 nm. IL-2 amounts were calculated based on readings of IL-2 standard.

### Results

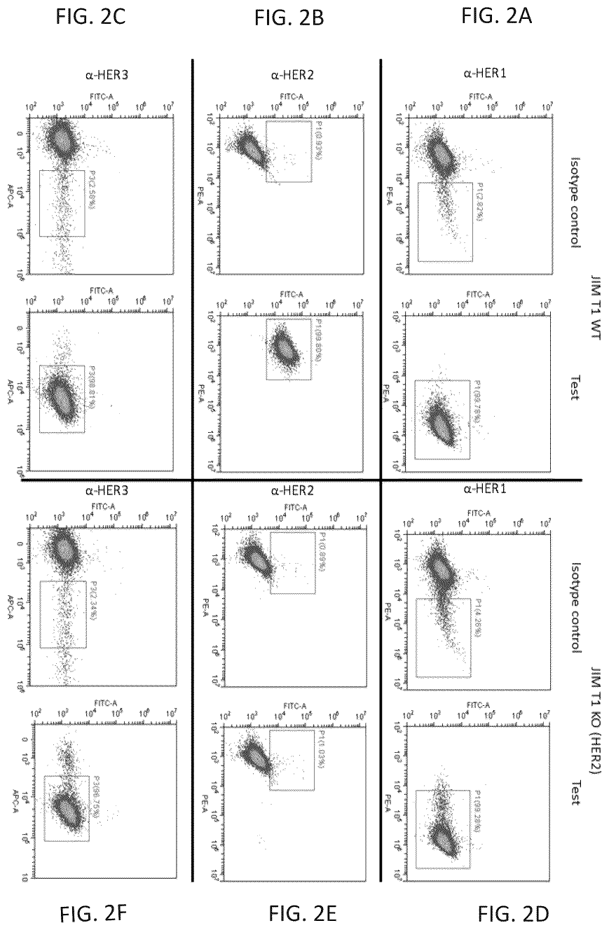

First, the expression of HER1, HER2 and HER3 in JIM T1 WT and KO (HER2 knockout with CRISPR) was tested by FACS. It was observed that JIM T1 WT expressed HER1, HER2, HER3 upon staining with their respective antibodies (FIGS. 2A-C). JIM T1 KO expressed HER 1 and HER3 (FIG. 2D-F).

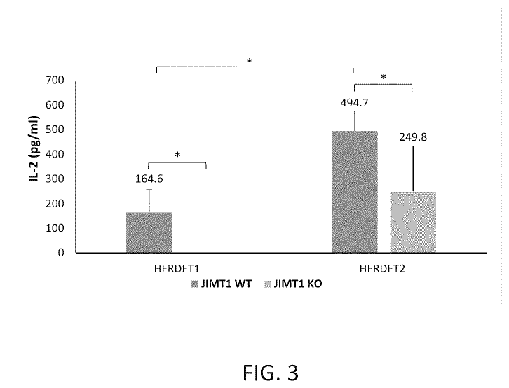

The response of HERDET1 and HERDET2 to JIM T1 cells that express various levels of all 4 HER receptors (Tanner et al., 2004) and to HER2-negative JIM T1 cells in which HER was knocked-out using CRISPR gene editing was evaluated. Looking at the expected pattern of HERDET1 response (FIG. 3) it is clear that knocking-out HER2 should reduce its response to near zero. Based on the literature-derived assessments (FIG. 1B), HERDET1 should strongly recognize the HER2-HER4 heterodimer and moderately recognized the HER2-HER3 heterodimer. HERDET1 should also recognize the homodimers of HER3 and HER4, but at a very low efficiency. Thus, knocking-out HER2, should strongly reduce HERDET1 response to HER2-KO JIM T1. These literature-based results are fully verified by the experimental results (IL-2 secretion by HERDET1 reporter) shown in FIG. 3. On the other hand, on the literature-derived assessments (FIG. 1B), HERDET2 should strongly recognize the heterodimers of HER2-HER3 and HER2-HER4 but also strongly recognize the HER3 and HER4 homodimers. Therefore, it is expected that HERDET2 response to cells expressing high levels of HER2+HER3+HER4 will not be affected from knocking out HER2. The experimental results detailed in FIG. 3 verify these assessments. HERDET1's high experimental response to JIMT1 cells is marginally affected when HERDET1 cells are exposed to HER2-KO JIM T1 target (FIG. 3).

FIG. 4 describes a panel of BW-HER standards that can be employed to calibrate and standardize the response of the HERDET AR array to clinical tumor samples.

### Example 2

### Evaluating an Effect of an Anti-Cancer Drug Targeting a Growth Factor Dependent RTK: Vascular Endothelial Growth Factor and its Receptor: VEGFA-VEGFR

### Scientific Background

Currently, inhibition of VEGFA interaction with vascular endothelial growth factor receptors (VEGFRs e.g., VEGFR1 and VEGFR2) is a common therapeutic strategy in oncology. This approach suppresses tumor angiogenesis and vasculature and thus reduces tumor growth. For example, Bevacizumab (brand name Avastin®) is a recombinant humanized mAb that blocks angiogenesis by inhibiting VEGFA interaction with VEGFR (Kazazi-Hyseni, Beijnen and Schellens 2010).

### Materials and Methods

Construction of BW-VEGFA reporters

The sequences employed are disclosed in FIG. 7.
Old Green (bold)—Signal peptide of light chain
Yellow (underline)—active part of a Ligand
Strong green (Bold underline)—Restriction Enzyme 1=RE1=SAL-I=DV in AA
Bright green (bold italics)—Myc tag 10AA peptide
Red (italics underline)—hinge (CD8a, mutated for Cyst)
Pink (bold underline italics)—murine Zeta inc. cyst in ectodomain (in bright green (small caps) marked the important AA)
   mIL-2 Secretion by BW-VEGFA with A549 in Presence of Avastin: Culturing and Co-Culturing of Cells

A549 cells were cultured in DMEM+10% FBS-NEAA-Sodium pyruvate-Hepes pH 7.3 10 mM and pen-strep. BW cells were cultured in RPMI+10% FBS++10% FBS-NEAA-Sodium pyruvate-Hepes pH 7.3 10 mM and pen-strep. During the co-culture assay, both cells were co-cultured (in Freestyle media+10% FBS. To some wells Avastin (5 μg/ml) was added during the co-culture.

100 ul of different amounts of BW cells (25000/50000 cells per well) in freestyle media+10% FBS was added to 96 well plate. 100 ul of different amounts of A549 in freestyle media+10% FBS (25000/50000 cells per well) was also added to the 96 well plate. 5 ug/ml of avastain prepared in freestyle media was added to the co-cultured cells and incubated for 16-18h at 37 C. IL-2 secretion was assessed by peforming standard ELISA as follows. ELISA plates were coated with purified anti-mouse IL2 (1:500 diluted in Na2HPO4 Coating buffer (0.1M pH=9)) and incubated overnight at 4° C. The plate was washed twice with PBST and blocked with 10% FBS. The plate was incubated for 2 h at room temperature, followed by washing with PBST twice. The experimental plate was centrifuged at 1200 rpm for 5 min, and the samples were added to the ELISA plate. The plate was incubated for 3 h at room temperature and then washed with PBST 6 times. Biotin anti-mouse IL2 (1:500 diluted in 2% FBS) was added to the plate and incubated for 1 h at room temperature. The plate was washed 6 times with PBST, and SA-HRP (1:750 diluted in 2% FBS) was added. After incubating the plate for 30 min. TMB was added, and the reading was recorded in the spectrophotometer plate reader at λ=650 nm. IL-2 amounts were calculated based on readings of IL-2 standard.

### Results

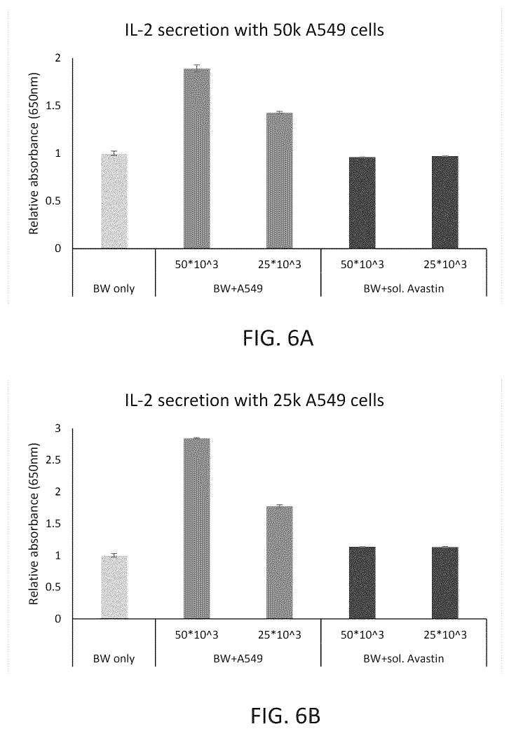

To functionally measure the interaction between reporter-associated VEGFA and bioavailable VEGFRs associated with a specific cancer sample, the present inventors developed artificial reporters (AR) expressing VEGFA based on the BW-based reporter system with chain zeta as the reporter element fused to a CD8-based hinge and the VEGFA (see FIG. 7 for sequences). A549 is a human NSCLC cell line expressing VEGFR2 that interacts with VEGFA. VEGFA-based BW artificial reporter was incubated with A549 cells and IL-2 secretion from BW-VEGFR cells was measured. Addition of Avastin (mAb-based blocker to VEGFA) significantly reduced the activation of AR-VEGFA to the background levels of IL-2 secretion (FIGS. 6A-B).

### Example 3

### Sequences and Construction of BW Reporters (NRG1a, NRG1b, BTC)

The study focused on creating and characterizing a system capable of detecting the presence and interaction of HER family homodimers and heterodimers on the membranes of target cancer cells. The system is similar to that mentioned in Example 1. As in Example 1, this system utilizes the BW reporter cell line, which was developed by selecting the active part of the ligands (53 amino acids of NRG1 alpha, 53 amino acids of NRG1 beta, and 81 amino acids of BTC) (FIG. 8A) and expressing the chimeric polypeptides in BW 5147 mouse thymoma cells (FIG. 8B). When the reporter cells bind the target receptors, they produce mIL-2, which acts as a measurable indicator of the binding activity (FIG. 8C). The following standards were produced in BW cells to express human her receptors: HER1, HER2, HER3, HER1+2, HER1+3, and HER2+3 (FIG. 8D). These standards were used to calibrate the system and evaluate its capacity to detect and distinguish between various HER family members.

For construction of the BW-reporters, The lentivirus for expression of the HERDETS was produced as follows: HEK293T cells were plated in 10 cm plates with DMEM containing 10% fetal bovine serum, pen-strep, L-glutamate, HEPES, non-essential amino acids, and sodium pyruvate, and cultivated till 90% confluence. Transfection with the NRG1a, NRG1b or BTC inserts in phage2 backbone was performed using JetPrime® reagent (Polyplus). After 2 days of infection, the lentivirus was collected and centrifuged at 500×g for 5 minutes and the supernatant was filtered. The virus was used to transduce mouse BW5147 thymoma cells, which were cultured in RPMI 1640 with 10% fetal calf serum, HEPES, sodium pyruvate, pen-strep, L-glutamine, and non-essential amino acids, along with 5 ug/ml of puromycin for selection. The cell lines were maintained at 37° C. with 5% CO2.

### Example 4

### Selection of the BW Reporters by FACS and ELISA

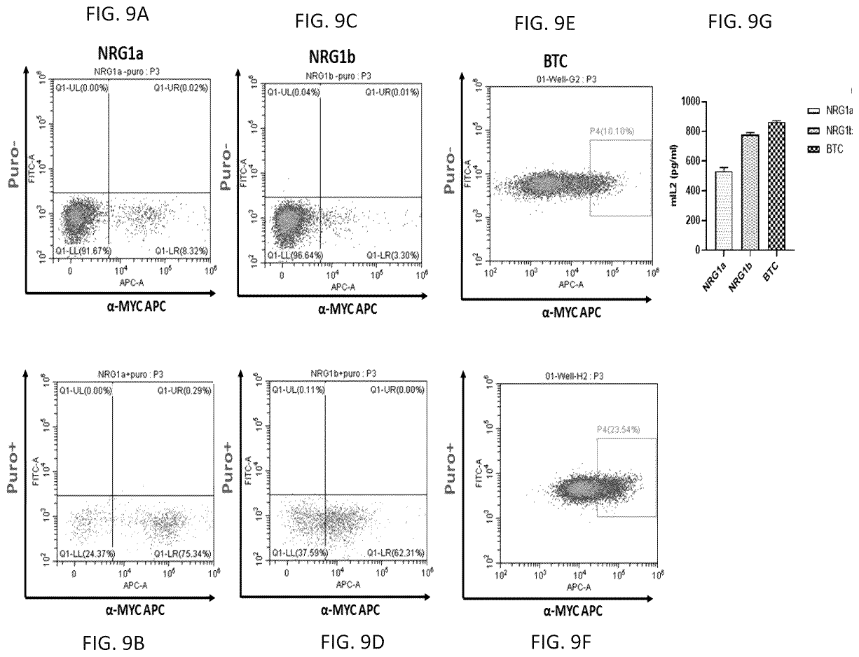

To confirm the successful construction of the BW reporters, the transduced BW cells were then checked for the expression of MYC through flow cytometry (FIGS. 9A-E). 50,000 cells/well were washed and seeded in 96 well plate. The cells were stained with α-MYC antibody (MABE282) (1:250) and incubated for 30 minutes on ice. The cells were washed and APC-conjugated AffiniPure F(ab′)2 fragment goat anti-mouse IgG+IgM (H+1) (1:200) was used as the secondary antibody for 30 minutes on ice. The cells were washed, and the dead cells were stained with DAPI. All the samples were analyzed in Beckman CytoFLEX flow cytometer. For gating, BW-WT was analyzed. The constructed BW-reporters were also tested for MYC expression through functional assay. A 96 U well plate was coated with 2.5 μg/ml of α-MYC (MABE282) and incubated for 1.5h at 37° C., 5% CO2. The antibody was washed twice with 1×PBS and 50,000 cells/well of the reporters were added and incubated for 16-18h at 37° C., 5% CO2. Standard mIL-2 ELISA was performed (FIG. 9F).

### Example 5

### Construction and Selection of BW Standards (HER1, HER2, HER3, HER1+2, HER1+3, HER2+3)

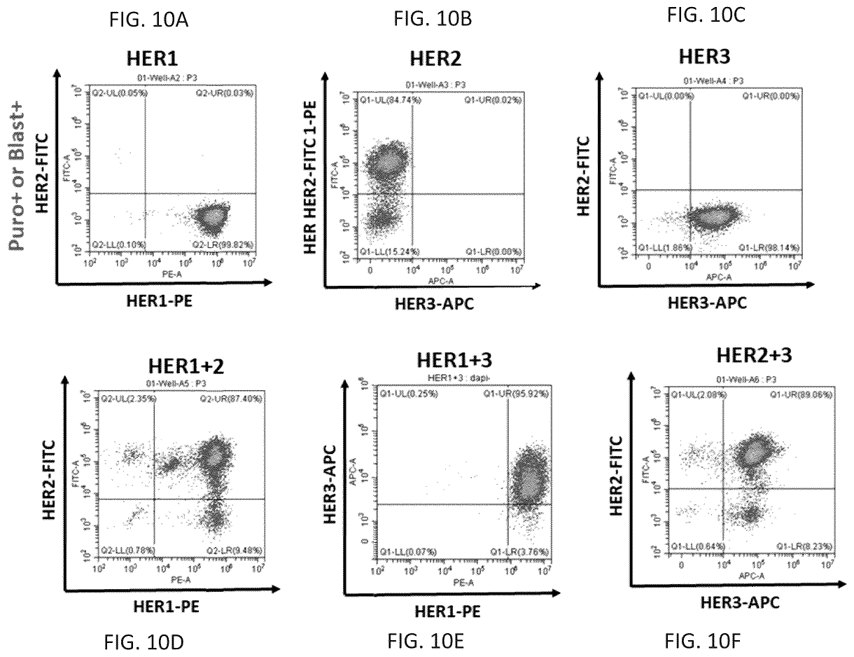

Similar to the BW reporters, BW standards expressing HER1, HER2, HER3, HER2+1, and HER2+3 were constructed and transduced to BW-WT. Co-transfection was performed where different HER receptors were to be expressed. The positive cells were selected through the addition of puromycin or blasticidin by sorting against PE-conjugated anti-human-HER1, FITC-conjugated anti-human-HER2, and APC-conjugated anti-human-HER3 antibodies with a final concentration of 100 μg/ml. FIGS. 10A-F show the successful construction of the BW standards (of the receptors).

### Example 6

### Binding Affinity of BW Reporters to BW Standards

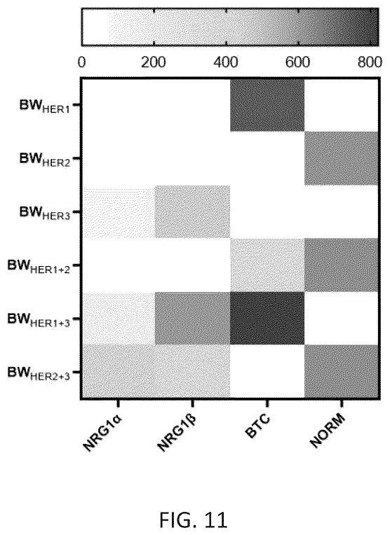

To check the binding of the BW reporters with the HER family, ELISA was performed where 50,000 cells/well of BW-standards were added along with 100,000 cells/well of BW reporters to 96 U well plate. Another BW construct (NORM) containing the trastuzumab domain was used as a normalizer which will bind HER2 (again bound to the zeta chain). The normalizer will report on the presence of HER2 monomers, honodimers and heterodimers. The plate was incubated at 37° C. with 5% CO₂ for 16-18h. After the incubation, the supernatant was analyzed for murine IL2 secretion by standard ELISA. The results show that the reporters had a unique affinity for specific HER dimers. NRG1b had a greater affinity for the HER3 homodimer, whereas NRG1a had a stronger affinity for the HER2+3 heterodimer. On the other hand, BTC showed a higher affinity to HER1+3 heterodimer than HER 1 homodimer (FIG. 11).

### Example 7

### Quantifying the Expression of HERs in BW Standards

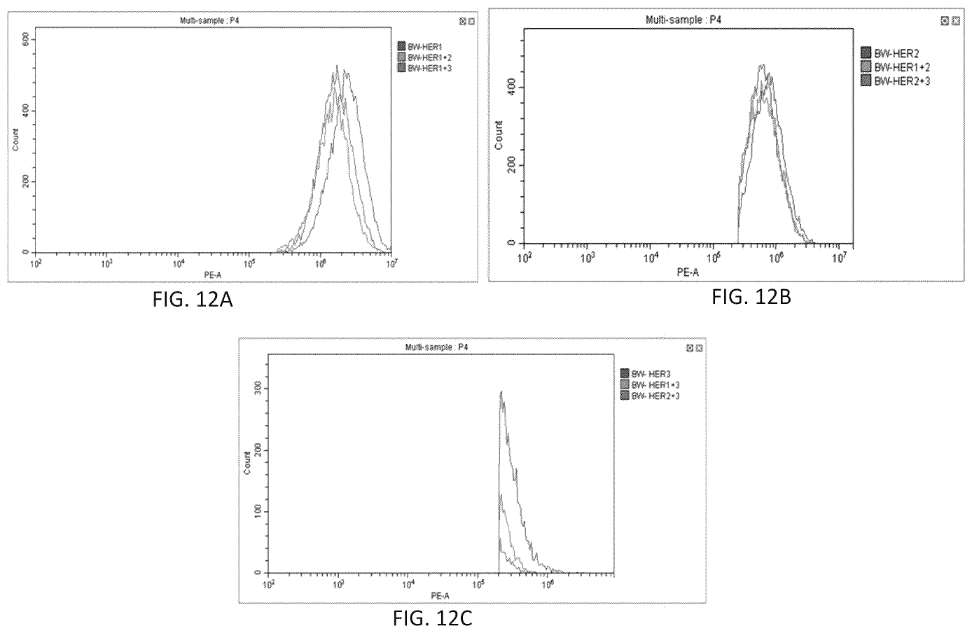

Since BW reporters showed a unique binding towards BW standards, to mitigate the possibility that binding is due to the overexpression of one HER in one BW standard than in the other we stained the BW standards with PE-conjugated anti-human EGFR, PE-conjugated anti-human CD340 (HER2), PE-conjugated anti-human HER3 and the samples were analyzed in a Beckman CytoFLEX flow cytometer. It was observed that HER1 expression was higher in BW-HER1 than BW-HER1+3 (FIG. 12A). However, BTC binds with higher binding to HER1+3 (FIG. 11). The HER2 expression in all the standards was observed to be the same (FIG. 12B). While the HER3 expression was highest in BW-HER3, followed by HER 1+3 and HER2+3 (FIG. 12C). However, NRG1a responds with a greater binding towards HER2+3, while NRG1b gives a higher response to HER 1 (FIG. 11). Therefore, it is possible to analyze the composition of HER 1, HER3 homodimers and HER1+2, HER1+3, HER2+3 heterodimers based on the relative response of NRG1a, NRG1b, and BTC reporters.

### Binding of BW Reporters to Cancer Cells Expressing HERs

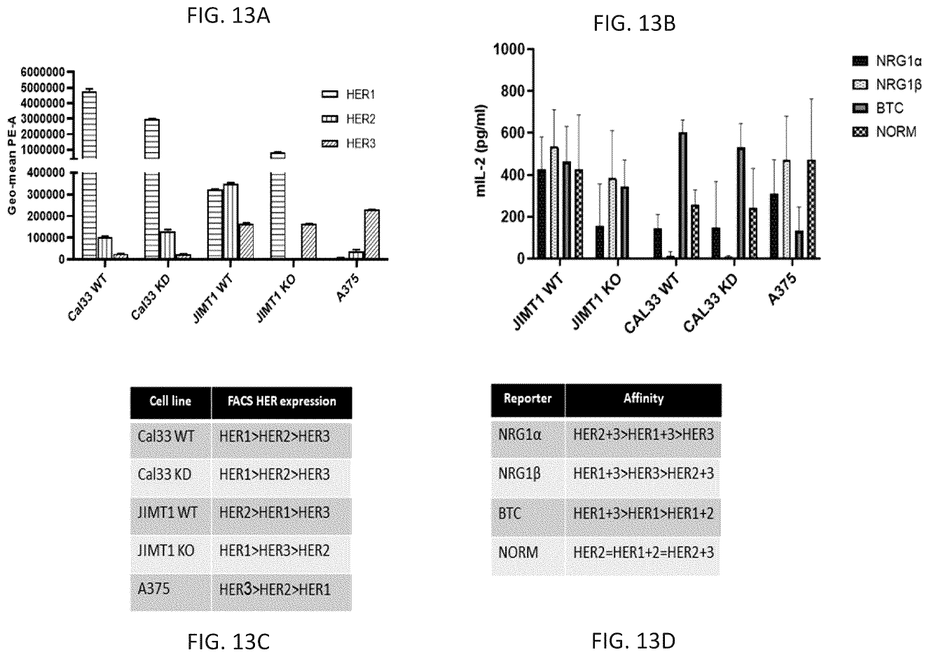

After determining the binding, the present inventors tested the reporter system on several cell lines. First, HER family's expression profile was tested in target cell lines. For this, 50,000 cells/well were stained with PE-conjugated anti-human EGFR, PE-conjugated anti-human CD340 (HER2), PE-conjugated anti-human HER3, and incubated for 30 minutes on ice with a final concentration of 100 ug/ml. PE-conjugated mouse IgG1,k was used as an isotype control. The cells were washed twice with 1×PAF, followed by the addition of DAPI to stain the dead cells. All the samples were analyzed in a Beckman CytoFLEX flow cytometer. For gating, isotype controls for each sample were analyzed. FIG. 13A shows that HER1 was highly expressed in Cal33 WT compared to Cal33 KD (knockdown for HER1). While HER2 was highly expressed in JIMT1 WT. No HER2 expression was observed in JIMT1 KO (knockout of HER2), as in Example 1. However, an increased expression in HER1 in JIMT1 KO was evident. Similarly, HER3 expression was high in A375. Moreover, FIG. 13B shows that Cal33 WT showed higher binding with BTC, while JIMT1 and A375 showed higher binding for NRG2b. mIL2 secretion with NORM was observed in all the cell lines except JIMT1 KO, depicting the presence of HER2 in all the cell lines mentioned except JIMT1 KO. Therefore, according to FIGS. 13C and D 3 scenarios are possible;

When HER1>HER2>HER3, possible dimer formation can be HER1+3>HER1>HER2+3, thus a higher response with BTC will be observed when HER1 is dominating; or alternatively when compared to NRG1a and NRG1b.

When HER3>HER2>HER1, possible dimer formation can be HER3>HER2+3>HER2+1, thus a higher response with NRG1b will be observed when compared to NRG1a and certainly when compared to BTC. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Others are Listed Throughout the Document

Arienti, C., Pignatta, S., & Tesei, A. (2019). Epidermal growth factor receptor family and its role in gastric cancer. Frontiers in oncology, 9, 1308.
Bunn Jr, P. A., Helfrich, B., Soriano, A. F., Franklin, W. A., Varella-Garcia, M., Hirsch, F. R., . . . & Chan, D. C. (2001). Expression of Her-2/neu in human lung cancer cell lines by immunohistochemistry and fluorescence in situ hybridization and its relationship to in vitro cytotoxicity by trastuzumab and chemotherapeutic agents. Clinical cancer research, 7 (10), 3239-3250.
Graus-Porta, D., Beerli, R. R., Daly, J. M., & Hynes, N. E. (1997). ErbB-2, the preferred heterodimerization partner of all ErbB receptors, is a mediator of lateral signaling. The EMBO journal, 16 (7), 1647-1655.
Jones, J. T., Akita, R. W., & Sliwkowski, M. X. (1999). Binding specificities and affinities of egf domains for ErbB receptors. FEBS letters, 447 (2-3), 227-231.
Luque-Cabal, M., García-Teijido, P., Fernández-Pérez, Y., Sánchez-Lorenzo, L., & Palacio-Vázquez, I. (2016). Mechanisms behind the resistance to trastuzumab in HER2-amplified breast cancer and strategies to overcome it. Clinical Medicine Insights: Oncology, 10, CMO-S34537.
Ma, S., Zhang, L., Ren, Y., Dai, W., Chen, T., Luo, L., . . . & Cao, B. (2021). Epiregulin confers EGFR-TKI resistance via EGFR/ErbB2 heterodimer in non-small cell lung cancer. Oncogene, 40 (14), 2596-2609.
Mitri, Z., Constantine, T., & O'Regan, R. (2012). The HER2 receptor in breast cancer: pathophysiology, clinical use, and new advances in therapy. Chemotherapy research and practice, 2012.
Muthuswamy, S. K., Gilman, M., & Brugge, J. S. (1999). Controlled dimerization of ErbB receptors provides evidence for differential signaling by homo- and heterodimers. Molecular and cellular biology, 19 (10), 6845-6857.
Oh, D. Y., & Bang, Y. J. (2020). HER2-targeted therapies-a role beyond breast cancer. Nature Reviews Clinical Oncology, 17 (1), 33-48.
Scharpenseel, H., Hanssen, A., Loges, S., Mohme, M., Bernreuther, C., Peine, S., . . . & Wikman, H. (2019). EGFR and HER3 expression in circulating tumor cells and tumor tissue from non-small cell lung cancer patients. Scientific reports, 9 (1), 1-9.
Thomas, R., & Weihua, Z. (2019). Rethink of EGFR in cancer with its kinase independent function on board. Frontiers in Oncology, 9, 800.
Tanner, M., Kapanen, A. I., Junttila, T., Raheem, O., Grenman, S., Elo, J., . . . & Isola, J. (2004). Characterization of a novel cell line established from a patient with Herceptin-resistant breast cancer. Molecular cancer therapeutics, 3 (12), 1585-1592.
Vu, T., & Claret, F. X. (2012). Trastuzumab: updated mechanisms of action and resistance in breast cancer. Frontiers in oncology, 2, 62.
Zheng, C., Shen, R., Li, K., Zheng, N., Zong, Y., Ye, D., . . . & Ma, Y. (2016). Epidermal growth factor receptor is overexpressed in neuroblastoma tissues and cells. Acta biochimica et biophysica Sinica, 48 (8), 762-767.
Keck, P. J., Hauser, S. D., Krivi, G., Sanzo, K., Warren, T., Feder, J., & Connolly, D. T. (1989). Vascular permeability factor, an endothelial cell mitogen related to PDGF. Science, 246 (4935), 1309-1312.
Leung, D. W., Cachianes, G., Kuang, W. J., Goeddel, D. V., & Ferrara, N. (1989). Vascular endothelial growth factor is a secreted angiogenic mitogen. Science, 246 (4935), 1306-1309.
Sa-Nguanraksa, D., & O-charoenrat, P. (2012). The role of vascular endothelial growth factor a polymorphisms in breast cancer. International journal of molecular sciences, 13 (11), 14845-14864.

1. A method of detecting presence and/or activity of a cancer-associated receptor in a cancer cell, the method comprising:
(a) contacting the cancer cell with a reporter cell comprising a polynucleotide encoding a chimeric polypeptide comprising an amino acid sequence of a ligand capable of binding a cancer-associated receptor, said amino acid sequence of said ligand being translationally fused to a heterologous amino acid sequence of a cell signaling module such that upon binding of the amino acid sequence of said ligand to said receptor, said cell signaling module is activated, wherein said ligand or cancer-associated receptor is not an immune checkpoint molecule;
(b) determining activation of said cell signaling module in the reporter cell, said activation being indicative of the presence and/or activity of the cancer-associated receptor or dimers or heterodimers thereof in the cancer cell.

2. A method of treating a subject diagnosed with cancer, the method comprising:
(a) detecting presence and/or activity of a cancer-associated receptor in a cancer cell of the subject according to claim 1; and
(b) treating the subject with an inhibitor of the receptor when presence or a predetermined threshold of activity of said cancer-associated receptor or dimers or heterodimers thereof is indicated or with another treatment modality when it is not indicated or absent.

3. (canceled)

4. The method of claim 2, wherein said inhibitor is a chimeric antigen receptor (CAR).

5. The method of claim 4, wherein said CAR is an effector immune cell expressing the polynucleotide encoding the chimeric polypeptide.

6. (canceled)

7. The method of claim 2, wherein said cancer-associated receptor is a receptor tyrosine kinase (RTK) optionally wherein said cancer-associated receptor is selected from the group of ErbB-1, ErbB-2, ErbB-3, ErbB-4 and VEGFA.

8. (canceled)

9. The method of claim 1, wherein said ligand is selected from the group consisting of NRG-1 alpha, NRG-1 beta and betacellulin (BTC).

10. The method of claim 1, wherein said detecting presence and/or activity of a cancer-associated receptor in a cancer cell comprises detecting presence and/or activity of plurality of cancer-associated receptors using a plurality of reporter cells each expressing at least one distinct chimeric polypeptide which binds said receptor or dimers or heterodimer thereof.

11. The method of claim 1, wherein said cancer-associated receptor is vascular endothelial growth factor receptor.

12. (canceled)

13. The method of claim 1, wherein said cell signaling module comprises a transmembrane domain and/or a cytoplasmic portion of a cell signaling receptor.

14. The method of claim 1, wherein said cell signaling module comprises a cytoplasmic portion of a cell signaling receptor.

15. The method of claim 13, wherein said cell signaling module comprises a transmembrane domain and/or a cytoplasmic portion of a receptor kinase.

16. The method of claim 15, wherein said receptor kinase is a tyrosine kinase or serine/threonine kinase.

17. The method of claim 13, wherein said cell signaling module comprises an adaptor molecule.

18. The method of claim 1, wherein said cell signaling module comprises a CD3 zeta chain.

19. (canceled)

20. The method of claim 2, wherein said determining activation is by analyzing a cytokine and/or an interleukin induced by said activation.

21-22. (canceled)

23. The method of claim 1, wherein the reporter cell is an immune cell or a non-cancerous cell.

24. (canceled)

25. The method of claim 1, wherein the reporter cell is null for expression of said cancer-associated receptor and/or ligand.

26. The method of claim 1, wherein the reporter cell is transformed to express a fluorescent or bioluminescent molecule upon activation of said cell signaling module.

27. The method of claim 1, wherein said contacting is in the presence of an inhibitor of said cancer-associated receptor.

28-33. (canceled)


SEQUENCE LISTING (55 sequences)

SEQ ID NO: 1 - protein, 5 aa, synthetic construct
  REGION 1..5: note = Linker peptide sequence
GGGGS

SEQ ID NO: 2 - protein, 20 aa, synthetic construct
  REGION 1..20: note = Linker peptide sequence
  VARIANT 6..10: note = may be absent
  VARIANT 11..15: note = may be absent
  VARIANT 16..20: note = may be absent
GGGGSGGGGSGGGGSGGGGS

SEQ ID NO: 3 - protein, 9 aa, synthetic construct
  REGION 1..9: note = Linker peptide sequence
GGGGSGGGG

SEQ ID NO: 4 - protein, 10 aa, synthetic construct
  REGION 1..10: note = Linker peptide sequence
GGGGSGGGGS

SEQ ID NO: 5 - protein, 14 aa, synthetic construct
  REGION 1..14: note = Linker peptide sequence
GGGGSGGGGSGGGG

SEQ ID NO: 6 - protein, 15 aa, synthetic construct
  REGION 1..15: note = Linker peptide sequence
  VARIANT 6..10: note = may be absent
  VARIANT 11..15: note = may be absent
EAAAKEAAAKEAAAK

SEQ ID NO: 7 - protein, 27 aa, synthetic construct
  REGION 1..27: note = Linker peptide sequence
  VARIANT 12..16: note = may be absent
  VARIANT 17..21: note = may be absent
  VARIANT 22..26: note = may be absent
AEAAAKEAAAKEAAAKEAAAKEAAAKA

SEQ ID NO: 8 - protein, 12 aa, synthetic construct
  REGION 1..12: note = Linker peptide sequence
AEAAAKEAAAKA

SEQ ID NO: 9 - protein, 46 aa, synthetic construct
  REGION 1..46: note = Linker peptide sequence
AEAAAKEAAAKEAAAKEAAAKALEAEAAAKEAAAKEAAAKEAAAKA

SEQ ID NO: 10 - protein, 5 aa, synthetic construct
  REGION 1..5: note = Linker peptide sequence
PAPAP

SEQ ID NO: 11 - protein, 18 aa, synthetic construct
  REGION 1..18: note = Linker peptide sequence
KESGSVSSEQLAQFRSLD

SEQ ID NO: 12 - protein, 14 aa, synthetic construct
  REGION 1..14: note = Linker peptide sequence
EGKSSGSGSESKST

SEQ ID NO: 13 - protein, 12 aa, synthetic construct
  REGION 1..12: note = Linker peptide sequence
GSAGSAAGSGEF

SEQ ID NO: 14 - protein, 20 aa, synthetic construct
  REGION 1..20: note = Linker peptide sequence
GGGGSGGGGSGGGGSGGGGS

SEQ ID NO: 15 - protein, 8 aa, synthetic construct
  REGION 1..8: note = Linker peptide sequence
GGGGGGGG

SEQ ID NO: 16 - protein, 6 aa, synthetic construct
  REGION 1..6: note = Linker peptide sequence
GGGGGG

SEQ ID NO: 17 - protein, 303 aa, synthetic construct
  REGION 1..303: note = artificial chimeric sequence
MDMRVPAQLLGLLLLWLSGARCSHLVKCAEKEKTFCVNGGECFMVKDLSNPSRYLCKCQPGFTGARCTENVPMKVGGGGSGGGGSGGGGSGGGGSDVEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 18 - protein, 303 aa, synthetic construct
  REGION 1..303: note = artificial chimeric sequence
MDMRVPAQLLGLLLLWLSGARCSHLVKCAEKEKTFCVNGGECFMVKDLSNPSRYLCKCPNEFTGDRCQNYVMASFGGGGSGGGGSGGGGSGGGGSDVEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 19 - protein, 300 aa, synthetic construct
  REGION 1..300: note = artificial chimeric sequence
MDMRVPAQLLGLLLLWLSGARCGHARKCNETAKSYCVNGGVCYYIEGINQLSCKCPVGYTGDRCQQFAMVNFGGGGSGGGGSGGGGSGGGGSDVEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 20 - protein, 299 aa, synthetic construct
  REGION 1..299: note = artificial chimeric sequence
MDMRVPAQLLGLLLLWLSGARCSHFNDCPDSHTQFCFHGTCRFLVQEDKPACVCHSGYVGARCEHADLLAVGGGGSGGGGSGGGGSGGGGSDVEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 21 - protein, 392 aa, synthetic construct
  REGION 1..392: note = artificial chimeric sequence
DMRVPAQLLGLLLLWLSGARCMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRRVDEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 22 - DNA, 21 bp, synthetic construct
  misc_feature 1..21: note = DNA sequence of cloning site
  source 1..21: mol_type = other DNA
gcggccgccggatccgaattc

SEQ ID NO: 23 - protein, 300 aa, synthetic construct
  REGION 1..300: note = artificial chimeric sequence
MDMRVPAQLLGLLLLWLSGARCMVAQVSITKCSSDMNGYCLHGQCIYLVDMSQNYCRCEVGYTGVRCEHFFLGGGGSGGGGSGGGGSGGGGSDVEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 24 - protein, 252 aa, synthetic construct
  REGION 1..252: note = artificial chimeric sequence
MDMRVPAQLLGLLLLWLSGARCNSDSECPLSHDGYCLHDGVCMYIEALDKYACNCVVGYIGERCQYRDLKWWELRGGGGSGGGGSGGGGSGGGGSDVEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNA

SEQ ID NO: 25 - protein, 348 aa, synthetic construct
  REGION 1..348: note = artificial chimeric sequence
MDMRVPAQLLGLLLLWLSGARCSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKGGGGSGGGGSGGGGSGGGGSDVEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 26 - protein, 330 aa, synthetic construct
  REGION 1..330: note = artificial chimeric sequence
MDMRVPAQLLGLLLLWLSGARCDGNSTRSPETNGLLCGDPEENCAATTTQSKRKGHFSRCPKQYKHYCIKGRCRFVVAEQTPSCVCDEGYIGARCERVDLFYGGGGSGGGGSGGGGSGGGGSDVEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 27 - protein, 336 aa, synthetic construct
  REGION 1..336: note = artificial chimeric sequence
MDMRVPAQLLGLLLLWLSGARCDLQEADLDLLRVTLSSKPQALATPNKEEHGKRKKKGKGLGKKRDPCLRKYKDFCIHGECKYVKELRAPSCICHPGYHGERCHGLSLGGGGSGGGGSGGGGSGGGGSDVEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 28 - protein, 311 aa, synthetic construct
  REGION 1..311: note = artificial chimeric sequence
MDMRVPAQLLGLLLLWLSGARCERSEHFKPCRDKDLAYCLNDGECFVIETLTGSHKHCRCKEGYQGVRCDQFLPKTDSILSDPGGGGSGGGGSGGGGSGGGGSDVEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 29 - protein, 334 aa, synthetic construct
  REGION 1..334: note = artificial chimeric sequence
MDMRVPAQLLGLLLLWLSGARCMGSSHHHHHHSSGLVPRGSHMGSMPTDHEEPCGPSHKSFCLNGGLCYVIPTIPSPFCRCVENYTGARCEEVFLPGSSIQTKSNLGGGGSGGGGSGGGGSGGGGSDVEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 30 - RNA, 25 nt, synthetic construct
  source 1..25: mol_type = other RNA; note = gRNA
caccgaactacctttctacggacgt

SEQ ID NO: 31 - RNA, 25 nt, synthetic construct
  source 1..25: mol_type = other RNA; note = gRNA
aaacacgtccgtagaaaggtagttc

SEQ ID NO: 32 - RNA, 25 nt, synthetic construct
  source 1..25: mol_type = other RNA; note = gRNA
caccgagaccgttggactcacgagt

SEQ ID NO: 33 - RNA, 25 nt, synthetic construct
  source 1..25: mol_type = other RNA; note = gRNA
aaacactcgtgagtccaacggtctc

SEQ ID NO: 34 - RNA, 25 nt, synthetic construct
  source 1..25: mol_type = other RNA; note = gRNA
caccgcagaacctgcaagtaatccg

SEQ ID NO: 35 - RNA, 25 nt, synthetic construct
  source 1..25: mol_type = other RNA; note = gRNA
aaaccggattacttgcaggttctgc

SEQ ID NO: 36 - RNA, 25 nt, synthetic construct
  source 1..25: mol_type = other RNA; note = gRNA
caccgtcatcgctcacaaccaagtg

SEQ ID NO: 37 - RNA, 25 nt, synthetic construct
  source 1..25: mol_type = other RNA; note = gRNA
aaaccacttggttgtgagcgatgac

SEQ ID NO: 38 - RNA, 25 nt, synthetic construct
  source 1..25: mol_type = other RNA; note = gRNA
caccgcagcgggagcccttacacat

SEQ ID NO: 39 - RNA, 25 nt, synthetic construct
  source 1..25: mol_type = other RNA; note = gRNA
aaacatgtgtaagggctcccgctgc

SEQ ID NO: 40 - DNA, 21 bp, synthetic construct
  source 1..21: mol_type = other DNA; note = Single strand DNA oligonucleotide
ctggttgttgtgaggggtaaa

SEQ ID NO: 41 - DNA, 20 bp, synthetic construct
  source 1..20: mol_type = other DNA; note = Single strand DNA oligonucleotide
taggtcccaagagggtctga

SEQ ID NO: 42 - DNA, 20 bp, synthetic construct
  source 1..20: mol_type = other DNA; note = Single strand DNA oligonucleotide
gcctgtgcccactataagga

SEQ ID NO: 43 - DNA, 21 bp, synthetic construct
  source 1..21: mol_type = other DNA; note = Single strand DNA oligonucleotide
gtggggatagaactgctaggg

SEQ ID NO: 44 - DNA, 20 bp, synthetic construct
  source 1..20: mol_type = other DNA; note = Single strand DNA oligonucleotide
agatcacaggtgggctctgt

SEQ ID NO: 45 - DNA, 20 bp, synthetic construct
  source 1..20: mol_type = other DNA; note = Single strand DNA oligonucleotide
gccactatgggagaaaggtg

SEQ ID NO: 46 - DNA, 20 bp, synthetic construct
  source 1..20: mol_type = other DNA; note = Single strand DNA oligonucleotide
ctatttcagccccactctgc

SEQ ID NO: 47 - DNA, 20 bp, synthetic construct
  source 1..20: mol_type = other DNA; note = Single strand DNA oligonucleotide
caatcttctgagggcgtctt

SEQ ID NO: 48 - DNA, 20 bp, synthetic construct
  source 1..20: mol_type = other DNA; note = Single strand DNA oligonucleotide
atgtgtaagggctcccgctg

SEQ ID NO: 49 - DNA, 20 bp, synthetic construct
  source 1..20: mol_type = other DNA; note = Single strand DNA oligonucleotide
ccctcccatgtcacctgtat

SEQ ID NO: 50 - protein, 53 aa, synthetic construct
SHLVKCAEKEKTFCVNGGECFMVKDLSNPSRYLCKCQPGFTGARCTENVPMKV

SEQ ID NO: 51 - protein, 53 aa, synthetic construct
SHLVKCAEKEKTFCVNGGECFMVKDLSNPSRYLCKCPNEFTGDRCQNYVMASF

SEQ ID NO: 52 - protein, 80 aa, synthetic construct
DGNSTRSPETNGLLCGDPEENCAATTTQSKRKGHFSRCPKQYKHYCIKGRCRFVVAEQTPSCVCDEGYIGARCERVDLFY

SEQ ID NO: 53 - protein, 640 aa, Homo sapiens
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 54 - protein, 590 aa, Homo sapiens [sequence omitted]

SEQ ID NO: 55 - protein, 178 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETNIA